(19) United States
(12) Patent Application Publication
(10) Pub. No.: US 2003/0101000 A1
(43) Pub. Date: May 29, 2003

(54) Family based tests of association using pooled DNA and SNP markers

(76) Inventors: Joel S. Bader, Stamford, CT (US); Pak Sham, London (GB)

Correspondence Address: MINTZ, LEVIN, COHN, FERRIS,, GLOVSKY and POPEO, P.C., One Financial Center, Boston, MA, 02111, US

(21) Appl. No.: 10/202,979

(22) Filed: Jul. 24, 2002

Related U.S. Application Data
(60) Provisional application No. 60/307,505, filed on Jul. 24, 2001.
(60) Provisional application No. 60/318,201, filed on Sep. 7, 2001.

(51) Int. Cl. G01N 33/48

(52) U.S. Cl. 702/20

(57) ABSTRACT

The invention relates to a system and methods for detecting an association in a population of individuals between a genetic locus or loci and a quantitative phenotype. In particular, the present invention relates to family based tests of association using pooled DNA. Disclosed are systems and methods for optimizing pooled tests as an explicit function of measurement error, and for family-based tests that eliminate stratification effects. Also disclosed are modules for identifying functional genetic variants and linked markers using systems and methods that are feasible with current-day instruments.

### RELATED APPLICATIONS

This application claims priority from U.S. provisional patent application serial No. 60/307,505, filed on Jul. 24, 2001, and serial No. 60/318,201, filed on Sep. 7, 2001, each of which is incorporated by reference in its entirety.

### FIELD OF THE INVENTION

The invention relates to a system and methods for detecting an association in a population of individuals between a genetic locus or loci and a quantitative phenotype, in particular the present invention relates to family based tests of association using pooled DNA.

### BACKGROUND OF THE INVENTION

Association tests of outbred populations are thought to have greater power than traditional family-based linkage analysis to identify the genetic variants contributing to complex human diseases. See, e.g, Risch and Merikangas, 1996; Ott 1999; Ardlie 2002. A genome scan based on allelic association would require approximately 100,000 markers, estimated by dividing the 3.3 gigabase human genome by the several kilobase extent of population-level linkage disequilibrium. See, e.g., Abecasis et al 2001; Reich et a/. 2001. Single-nucleotide polymorphisms (SNPs) occur at sufficient density to provide a suitable marker set. See, e.g., Collins et al 1997. Furthermore, SNPs in coding and regulatory regions have additional value as potential functional variants.

Individual genotyping remains prohibitively expensive for a genome scan. One method to reduce associated costs is to pool DNA from individuals with extreme phenotypic values and to measure the allele frequency difference between pools. See, e.g., Barcellos et al., 1997; Daniels et al., 1998; Fisher et al., 1999; Hill et al., 1999; Shaw et al., 1998; Stockton et al, 1998; Suzuki et al, 1998. Initial attention focused on pooled designs for dichotomous traits and case-control studies. See, e.g., Risch and Teng 1998.

More recently, pooled tests have been discussed for quantitative traits, which is a more appropriate model for diseases such as obesity and hypertension. In the absence of experimental error, the existing “optimal” design for an unrelated population is to compare frequencies between pools of the most extreme 27% of individuals ranked by phenotypic value, retaining 80% of the information of individual genotyping. See, e.g., Bader et al., 2001.

Experimental sources of error, which are primarily allele frequency measurement errors, degrade the test power. See, e.g., Jawaid et al., 2002. Therefore, one drawback of existing systems is a lack of methods for estimating test power that explicitly includes allele frequency measurement error for pooled tests.

Population stratification poses a second challenge to practical use of pooled tests for human populations. However, current genomic control methods, developed to reduce stratification effects in genotype-based association tests (see, e.g, Devlin and Roeder 1999; Pritchard and Rosenberg 1999; Pritchard et al 2001; Zhang and Zhou, 2001), are not directly applicable to pooled tests.

Existing systems lack the methodology to optimize pooled DNA test designs that are robust to stratification. Yet another drawback of existing systems is a lack of methods that permit the optimization of test design as a function of known parameters, and to provide a bridge to experimentalists seeking practical guidance for whether to attempt and how to perform pooled association tests. A need exists for ways to fill these voids.

### SUMMARY OF THE INVENTION

Included in the invention are methods and systems that overcome these and other drawbacks in existing systems by providing a system for family based association testing for quantitative traits using pooled DNA. The system of the present invention includes various methodologies, such as optimizing pooled DNA test designs including one or more tests robust to stratification; permitting the optimization of a test design as a function of known parameters; enabling a user seeking practical guidance for whether to attempt and how to perform pooled association tests; and estimating test power that explicitly includes allele frequency measurement error.

In one embodiment, the invention detects an association in a population of unrelated individuals between a genetic locus and a quantitative phenotype, wherein two or more alleles occur at the locus, and wherein the phenotype is represented by a numerical phenotypic value whose range falls within pre-determined numerical limits.

In another embodiment, the invention comprises at least one module for obtaining the phenotypic value for each individual in the population and determining the minimum number of individuals from the population required for detecting an association using a preferred non-centrality parameter.

In yet another embodiment, the invention comprises at least one module for selecting a first subpopulation of individuals having phenotypic values that are higher than a predetermined lower limit and pooling DNA from the individuals in this first subpopulation. In a parallel embodiment, the invention includes selecting a second subpopulation of individuals having phenotypic values that are lower than a predetermined upper limit and pooling DNA from these individuals in the second subpopulation.

In a further embodiment, the invention measures the frequency of occurrence of each allele at a given locus for one or more genetic loci.

In another embodiment, the invention measures the difference in frequency of occurrence of a specified allele between pools of two sub-populations for a particular genetic locus and determines that an association exists where the allele frequency difference between the pools is larger than a predetermined value.

In an additional embodiment, the invention includes at least one module for classifying individuals in a population. In one aspect of the invention, the classes are based on an age group a gender, a race or an ethnic origin. In another aspect of the invention, all members of a class are included in the pools. In a contrasting aspect of the invention, fewer than all members of a class are included in the pools. The systems and methods of the present invention for family based association tests for quantitative traits using pooled DNA are advantageous for detecting associations between a genetics locus or loci and a phenotype of complex diseases. Complex diseases include, but are not limited to, e.g., cancer, cardiovascular disease, and metabolic disorders.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 is a flow chart illustrating one embodiment of the invention, wherein a family based association test for quantitative traits using pooled DNA begins by selecting portions of a population according to a predetermined value for a trait (10), pooling the genetic material from these portions of the population (15), measuring the frequency of alleles with methods including mass spcctrophotometry (“mass spec”), real-time quantitation polymerase chain reactions (RTQ-PCR”), and/or various sequencing methods (“pyro”) (20) known to those skilled in the art, and displaying the resulting association detected between the input gene locus and phenotype (25).

FIG. 2 is a flow chart illustration for family based association tests for quantitative traits using pooled DNA in a two-stage design.

FIG. 3 illustrates a system architecture for family based association tests for quantitative traits using pooled DNA.

FIG. 4 illustrates a system of the invention implemented in an integrated genotyping device.

FIG. 5 illustrates a user interface for the inventive system implemented in an integrated genotyping device.

FIG. 6 graphically illustrates the information retained by a pooled test, expressed as a fraction of the theoretical maximum from individual genotyping, as a function of the pooling fraction for three family sizes, namely sib-quads, sib-pairs, and unrelated individuals.

FIGS. 7A-7F graphically illustrate the information related to various allele frequencies in a population retained as a function of the pooling fraction for between-family tests (FIGS. 7A-7C) and within-family tests (FIGS. 7D-7F) for a population of 500 sib-pairs (1000 individuals).

FIGS. 8A and 8B graphically illustrate the optimal pooling fraction (FIG. 8A) and the information retained (FIG. 8B) from exact numerical calculations (solid line) and an analytical fit (dashed line) as a function of the normalized measurement error K.

FIG. 9 is a flow-chart for designing a two-stage study.

### DETAILED DESCRIPTION

1. Definitions

Glossary of Mathematical Symbols

X quantitative phenotypic value of an individual

Xᵢ quantitative phenotypic value of sib i, where i=1 or 2 for sib-pairs

X_{±} (X₁X₂)/2

r phenotypic correlation between sibs

Aᵢ allele inherited at a particular locus. For a bi-allelic marker, i=1 or 2

G genotype of a locus, e.g., either A₁A₁, A₁A₂, or A₂A₂ for a bi-allelic market

Gᵢ genotype for sib i, where i=1 or 2 for sib-pairs

P(G) genotype probability

P(G₁,G₂) joint sib-pair genotype probability

f(X₁,X₂) joint sib-pair phenotype probability distribution

f[X₁,X₂|G₁,G₂] joint sib-pair phenotype probability distribution conditioned on genotypes

p frequency of allele A₁ in a population

q frequency of the remaining alleles, where q=1−p

pᵢ frequency of allele A₁ in sib i, e.g., either 1, 0.5, or 0 for an autosomal marker

p_{±} (p₁±p₂)/2

a half the difference in the shift in the mean phenotypic value of individuals between genotype A₁A₁ compared to A₂A₂

d difference in the mean phenotypic value between individuals with genotype A₁A₂ compared to the mid-point of the means value for A₁A₁ and A₂A₂

μ mean phenotypic shift due to the locus, equal to a(p−q)+2pqd

σ_{A}² additive variance of phenotype X due to the genotype G

σ_{D}² dominance variance due to the genotype G

σ_{R}² residual phenotypic variance, where σ_{A}²+σ_{D}²+σ_{R}²=1

N total number of individuals whose DNA is available for pooling

n number of individuals selected for a single pool

ρ pooling fraction defined as n/N

p_{U},p_{L} frequency of allele A₁ in the upper (U) or lower (L) pool

T test statistic, which is expected to be close to zero when the genotype G does not affect the phenotypic value and is expected to be non-zero when individuals with genotypes A₁A₁, A₁A₂, and A₂A₂ have different mean phenotypic values. As formulated here, T has a normal distribution with unit variance. Under the null hypothesis that CA (2pq)^{1/2}[a−(p−q)d] is zero, the mean of T is zero. Under the alternative hypothesis that GA is non-zero, the mean of T is also non-zero.

σ₀² variance of n^{1/2} (p_{U}−p_{L}) under the null hypothesis

σ₁² variance of n^{1/2} (p_{U}−p_{L}) under the alternative hypothesis

Φ(z) cumulative standard normal probability, the area under a standard normal distribution up to normal deviate z

z_{α} normal deviate corresponding to an upper tail area of α, defined as Φ(z_{α})=1−60

α type I error rate (false-positive rate). For a one-sided test, T>z_{α} corresponds to statistical significance at level α, typically termed a p-value. A typical threshold for significance is a p-value smaller than 0.05 or 0.01. If M independent tests are conducted, a conservative correction that yields a final p-value of α is to use a p-value of α/M for each of the M tests.

β type II error rate (false-negative rate). The power of a test is 1−β.

As used herein, when two individuals are “related to each other”, they are genetically related in a direct parent-child relationship or a sibling relationship. In a sibling relationship, the two individuals of the sibling pair have the same biological father and the same biological mother.

As used herein, the term “sib” is used to designate the word “sibling.” The sibling relationship is defined above. The term “sib pair” is used to designate a set of two siblings.

The members of a sib pair may be dizygotic, indicating that they originate from different fertilized ova. A sib pair includes dizygotic twins.

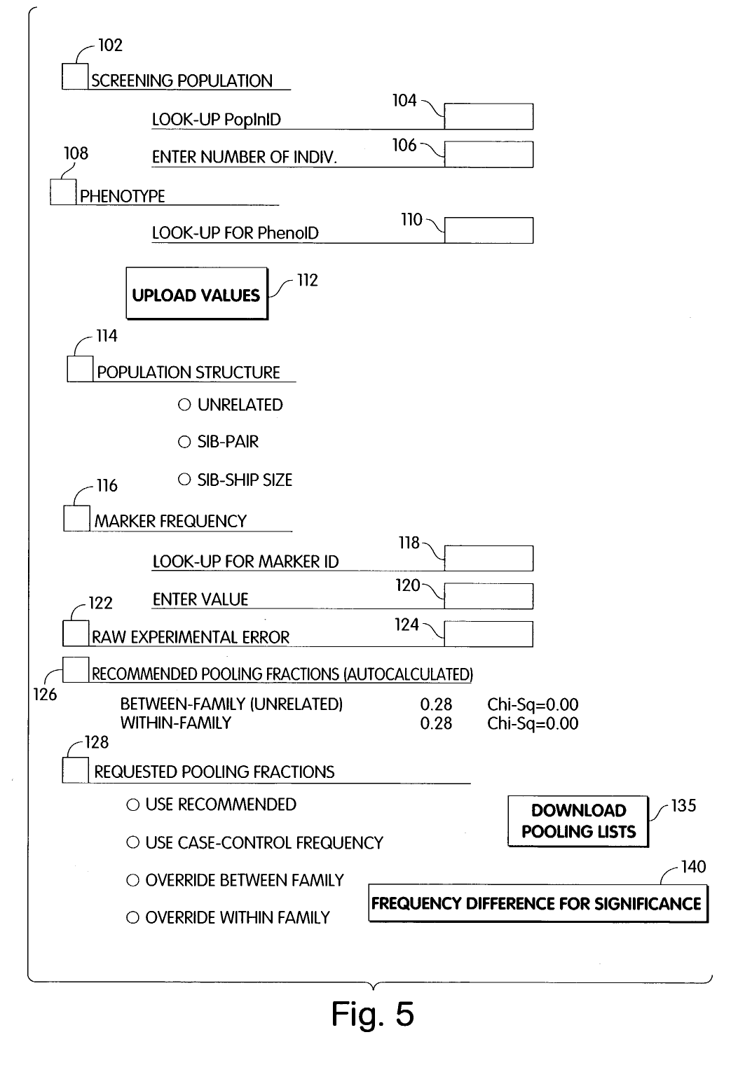

The term “quantitative trait locus”, or “QTL”, is used interchangeably with the term “gene” or related terms, including alleles that may occur at a particular genetic locus. Contemplated as within the scope of the invention is a “selection module”, which encompasses the term selection means, and which can be a first processor readable program code. In one embodiment, a “selection module” includes a processor readable routine or program that would select at least one individual with a pre-determined phenotypic value. These processor readable routines or programs would communicate with one or more user interfaces, preferably a graphical user interface (e.g. FIG. 5). A user would be able to enter phenotypic values in one or more interfaces that would cause a processor to execute a program for selecting individuals from one or more phenotypic databases. The phenotypic database could comprise at least one unique individual identification number and one or more phenotypic values for each individual. In a specific embodiment, a phenotypic database would include other modifiable user input information that is related to a phenotype of one or more individuals. In certain embodiments, selection of individuals would be performed automatically without user intervention, based on pre-determined routines. In a parallel embodiment, phenotypic data that is input into the selection module analysis is derived from a preexisting database. Computer readable program code would be used to select individuals with at least one pre-determined phenotypic value.

Also within the scope of the invention is a “pooling module”, which alternatively encompasses the term pooling means, and which can be a second processor readable program code. In a given embodiment, a “pooling module” provides genetic materials from selected individuals that would be pooled in a tube commonly used in a laboratory for handling nucleotides or proteins. Alternatively, a laboratory based automizer would be used to pool nucleotides or proteins, wherein a laboratory based automizer are operably controlled by a processor and includes programmable features for pooling nucleotides or proteins. Each pool could be hybridized with one or more genetic markers in the laboratory. Each marker could correspond to at least one allele. Hybridization would be performed by any method known to one skilled in the art. Information obtained from the results of a hybridization could be stored as one or more genotypic databases. A genotypic database could also comprise annotations for each marker. In a parallel embodiment, a pooling module is a computer readable program code, and what is pooled is the data obtained from a selected individual's genotype.

Genotypic and phenotypic databases of the present invention could be proprietary, open source (e.g., GenBank, EMBL, SwissProt), or any combination of proprietary and open source databases. Furthermore, genotypic and phenotypic databases of the present invention could be true object oriented, true relational or hybrid of object and relational databases. Which genotypic or phenotypic database to use, or whether to generate a genotypic or phenotypic database de novo, would be well known to one skilled in the art.

Also contemplated as within the scope of the invention is a “measuring module”, which encompasses the term measuring means, and which can be a third processor readable program code. In one embodiment of a “measuring module,” a user is able to instruct the processor to measure allele frequency of one or more selected markers in one or more selected group of individuals. Processor readable routines or programs would cause the processor to measure allele frequency by obtaining the genotypic data of one or more markers from one or more genotypic databases and calculate the allele frequency using at least one programmable formula. In some embodiments, a user would be able to intervene and add new variables to a programmable formula. In a given embodiment, the genotypic database is derived from the results of the selection module and/or the pooling module. In an alternative embodiment, the information or genetic material input into the selection module and/or the pooling module is derived from a preexisting genotypic database.

Included within the scope of the invention is an “association detection module”, which encompasses the term association detection means, and which can be a fourth processor readable program code. In this aspect of the invention, at least one processor readable routine or program would cause the processor to detect an association between at least one genetic locus and at least one phenotype by measuring the allele frequency difference between the pools. This detection could be performed by one or more user selectable programmable formula(s). In certain embodiments, association detection would be performed automatically without user intervention, and would be based on pre-determined routines.

Also included within the scope of the invention is a “reporting module”, which encompasses the term reporting means, and which can be a fifth processor readable program code. According to another aspect of the invention, the results of the association detection, described above, would be reported to a user. A user could optionally design and select a report and output it in a user preferred presentation format. The user would be able to instruct the processor to store one or more reports.

2. Aspects of the Invention

The present invention relates to systems and methods for detecting an association in a population of individuals between a genetic locus or loci and a quantitative phenotype. In particular the present invention relates to family based tests of association using pooled DNA.

While SNP-based marker sets and population-level DNA repositories are approaching sufficient size for whole-genome association studies, individual genotyping remains very costly. Pooled DNA tests are a less costly alternative, but uncertainty about loss of test power due to allele frequency measurement errors and population stratification hinders their use. According to one embodiment, the present invention may optimize pooled tests as an explicit function of measurement error, and may present family-based tests that eliminate stratification effects. According to another embodiment, the present invention may identify functional genetic variants and linked markers that are feasible with current-day instruments.

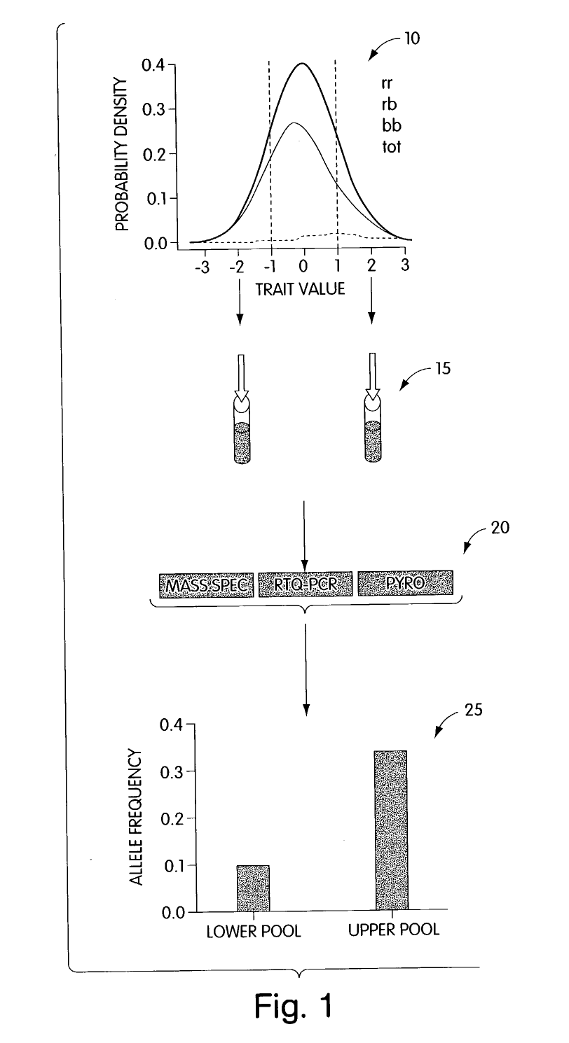

According to one embodiment, the present invention may associate a genetic locus having two or more alleles with the presence of one or more phenotypes. According to one aspect, the present invention comprises a selection module, a pooling module, a measuring module, an association detection module, and a reporting module. As embodied in FIG. 1, one aspect of the invention detects association of a genetic locus with a quantitative phenotype and identifies QTLs by tests of pooled DNA. In one embodiment, individuals with extreme phenotypic values are selected. For example, in FIG. 1 box 10, those individuals having a trait (phenotypic) value greater than one (>1) and those individuals having a trait (phenotypic) value less than one (<1) may be selected for the detection of association between genotype and phenotype. In some embodiments selected, individuals may be chosen from disease cases compared to normal controls (no disease). In FIG. 1, box 15, genetic materials from individuals in each of the selected groups are pooled. Examples of genetic materials may include, but are not limited to, DNA, proteins or their products, derivatives, homologs, analogs, or fragments. In FIG. 1, box 20, the frequency of alleles in each pool may be measured by plurality of measuring devices. In one embodiment, allele frequency is measured in terms of the frequency of occurrence of nucleotide fragments (e g DNA) using nucleotide hybridization methods (e.g. southern blotting) or other analytical devices (e.g. real-time PCR, Microarray chips). In another embodiment, allele frequency may be measured in terms of the frequency of occurrence of a peptide fragment (e.g. protein) using protein hybridization methods (e.g. western blotting) or other analytical devices (e g mass spectrophotometry). Allele frequency may be measured for each pool of selected individuals. In FIG. 1, box 25, analysis of the experimental results, preferably in terms of the allele frequency difference between pools, may be performed to detect the association an allele and a phenotype. FIG. 1, box 25, depicts a graphic output report of one such analysis.

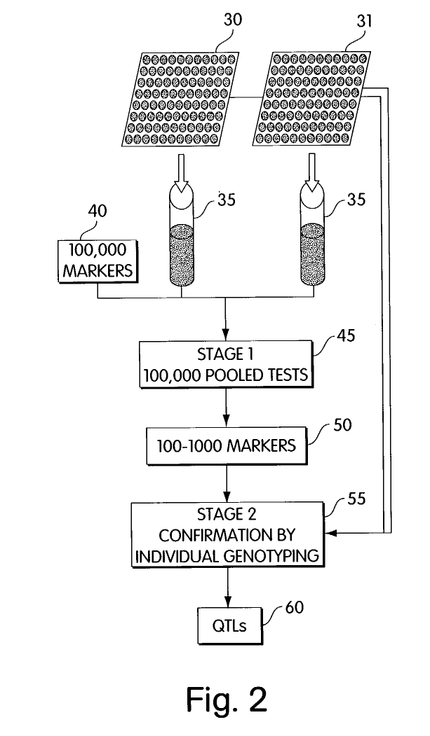

As illustrated in FIG. 2, the detection of an association may be performed in at least two stages. In one embodiment, the individuals may be selected from disease cases 30 and controls 31. In another embodiment, the individuals with extreme phenotypic values may be selected as illustrated in FIG. 1, item 10. Genetic materials of selected individuals may be pooled 35 and hybridized preferably with about 100,000 markers 40. Contemplated numbers of selected individual to be input may be about 10, about 50, about 100, about 500, about 1000, about 5000, about 10,000, about 50,000, about 100,000, about 500,000, or about 1 million markers. The first stage 45 may use pooled tests to reduce a marker set (possibly a whole-genome fine map) by 100-fold to 1000-fold. In the second stage 55, a reduced number of markers may be genotyped against the original sample to confirm the pooled test results. According to one embodiment, the smallest QTL 60 effect that may be detected in such a two-stage screen will result where a p-value is 0.001 and has a ⁹⁰% power for the first stage and where p 0.00001 (one false-positive in 100,000 tests) and has 80% power for the second stage. These results may assume a low-prevalence of disease and access to about 500 cases and about 500 controls. Contemplated numbers of individuals in the case or control groups may be about 10, about 50, about 100, about 500, about 1000, about 5000, about 10,000, about 50,000, about 100,000, about 500,000, or about 1 million individuals. The relative risk is assumed to be a multiplicative and may be depicted for the heterozygote. The relative risk for the protective allele homozygote may be defined to be one (1).

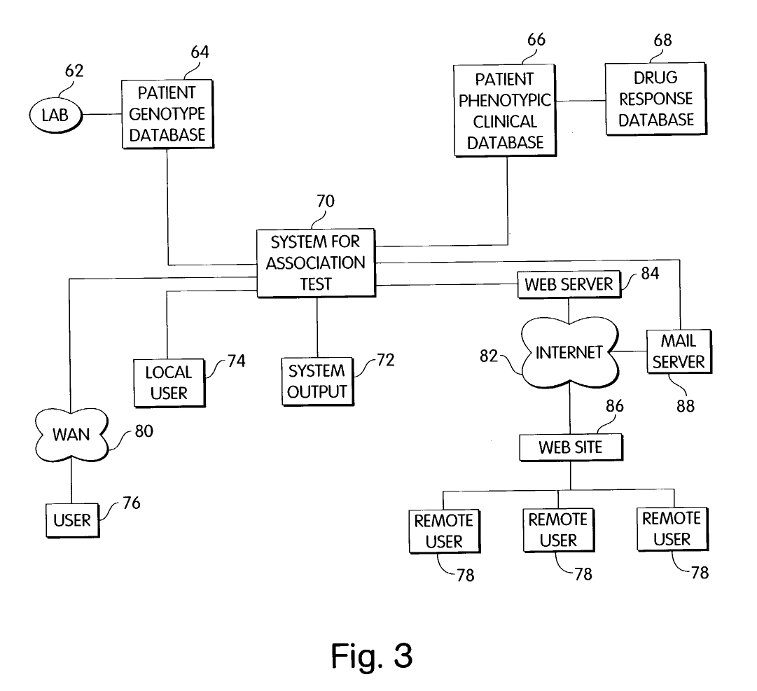

According to another aspect of the invention, analysis of association between one or more genetic locus or loci and one or more phenotypes may be carried out using a computer-based system. As illustrated in FIG. 3, a system for an association test 70 may have a means to access and retrieve genotypic data from a patient genotype database 64 and phenotypic data from a patient phenotypic clinical database 66. The patient genotype database 64 may be derived from genotypic data obtained from laboratory analysis 62. Alternatively, phenotypic clinical database 66 from patients may be obtained from data from clinical trails. The patient phenotypic clinical database may be connected to a drug response database 68. The results of the association test performed by the system 70 may be stored in a system output 72. The system 70 may be accessed by a local user 74 and/or a user 72 in a WAN (Wide Area Network) 80. The system 70 may also be accessed by a remote user 78 using the internet 82 through a web server 84. A website 86 may facilitate access and authorization to remote a user 78. The system 70 may also communicate with a remote user 78 by electronic mail through a mail server 88. The system 70 may be compatible with any operating system, hardware and software known to one skilled in the art.

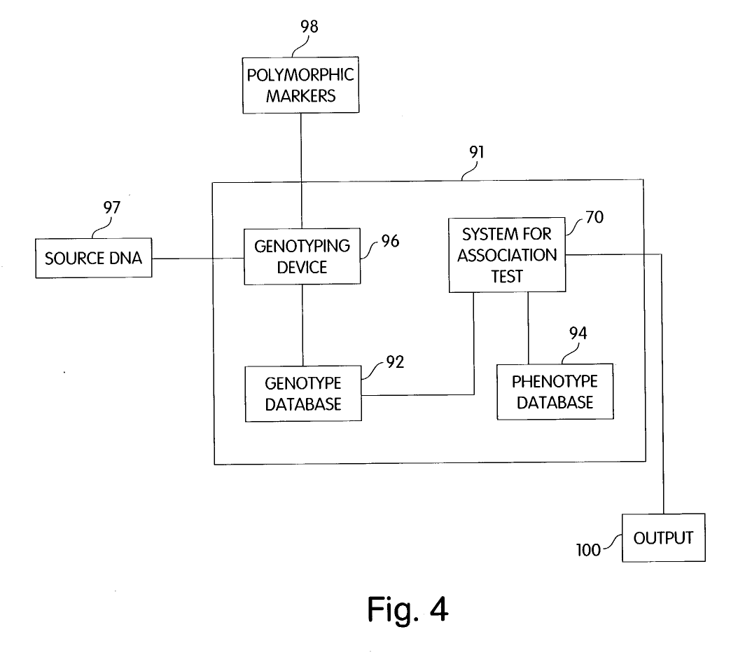

As illustrated in FIG. 4, the system 70 may also be implemented in an integrated device 92 for genetic analysis. The integrated device 92 may also comprise a genotyping device 96, a genotype database 92, and a phenotype database 94. The genotyping device may use source DNA 97 as a template or a probe for hybridization. The source DNA 97 may comprise DNA samples from a plurality of individuals. The genotyping device 96 may also use polymorphic markers 98 as a probe or template for hybridization. The polymorphic markers may preferably be SNP (Single Nucleotide Polymorphism) markers. The system 70 may optionally send the results of an analysis of an association test to an output 100 for storing, printing, etc.

Optimizing the selection threshold is crucial for good sensitivity and selectivity, and requires an understanding of the sources of variation in the measured allele frequency difference between pools. According to one object of the invention, the sources of variation may be due to the presence of unequal amounts of DNA contributed by various selected individuals to a pool prepared for analysis, from raw measurement error, and/or from sampling errors for a finite population.

FIG. 5 illustrates a user interface for auto-calculating an optimized pooled test design. The user interlace may have one or more frames and a plurality of buttons preferably in a graphical user interface for inputting, outputting and analyzing genotypic and phenotypic information. In one embodiment, a user interface may have panels for screening a population 102, a phenotype 108, a population structure 114, a marker frequency 116, a raw experimental error 122, a recommended pooling fractions 126, and/or a requested pooling fraction 128. In addition, the user interface may have controls for uploading values 112 and downloading pooling lists, and a window for output 140.

In a screening population module 102, a user may enter the identification information about the screening population in a PopInID window 104. A user may also specify the number of individuals in the population. A user interface module for phenotype related information 108 may have windows for entering identification information in the PhenoID window 110. Population and phenotypic information may be uploaded using upload value control 112. In a population structure panel 104, a user may input the type of population being used in the experiment or analysis. In one embodiment, the types of populations used may include unrelated, sib-pair and/or sib-size population. The marker frequency panel 116 may have windows 118 for entering a marker ID. A user may also enter values for the marker frequency using an alternative window 120. Raw experimental error may be specified using window 124. Panel 126 may provide for automatically calculating the recommended pooling fractions. Possible auto-calculated information may be optimized for between-family and within-family tests. Requested pooling fraction panel 128 may provide a user selectable features such as the use recommended, the use case control frequency, an override between-family option, and an override within-family option. A user may provide specific values for these features. A downloading pooling list control 135 may download the pooling list. An output 140 may provide the frequency difference for significance determination.

According to one embodiment of the invention, optimized designs for pooled DNA tests may be conducted on a population of N/s families, where each has a sibship of size (i.e., N total individuals). The genotypic correlation within a sibship is denoted r, with typical values of ¼, ½, and 1 for half-sibs, full-sibs, and monozygotic twins, respectively. Sibships may also represent inbred lines. In this case, r is the genetic correlation within each line. In general, sibs in different families may be assumed to have uncorrelated genotypes.

According to another embodiment of the invention, to conduct a pooled DNA test for association of a particular allele A₁ with a quantitative trait, individuals may be selected for an upper pool, which would include individuals with the higher phenotypic values, and a lower pool, which would include individuals with the lower phenotypic value, using designs reminiscent of selection strategies for optimizing breeding value and for QTL mapping. One advantage of the invention is a balanced design in which each pool may have fN individuals, where f≦0.5 is defined as the pooling fraction. Balanced designs may be favored when high and low phenotypes are treated symmetrically.

In one embodiment, unrelated individuals (s=1), in which the fN individuals having highest and lowest phenotypic values, may be selected for the upper and lower pools, respectively. In another embodiment, between-family groups, wherein all s sibs from the fN/s families have the highest and lowest mean phenotypic values, may be selected for the upper and lower pools. In yet another embodiment, within-family groups, in which the s′ sibs have the highest and lowest phenotypic values within each family, may be selected for the upper and lower pools, yielding a pooling fraction f=s′/s. In a further embodiment, within-family tests will pre-select discordant families, where the fraction f′ of families with the greatest within-family phenotypic variance are selected, and wherein the variance (Var) may be estimated according to the relation: Var=Σₛ(Xₛ−\overline{X})², where Xₛ is the phenotype of sib s and \overline{X} is the family mean. For within-family tests of discordant families, the extreme high and low sib within each selected family may be selected for the upper and lower pool for a final pooling fraction f=f′/N.

A preferred statistic for a two-sided test for each design described above is:

$${Z}^{2} = \frac{{( {\hat{p}}_{U} - {\hat{p}}_{L} )}^{2}}{Var ( {\hat{p}}_{U} - {\hat{p}}_{L} )} , \qquad [ 1 ]$$

where the estimated frequency of allele A₁ in the upper and lower pools is denoted \hat{p}_{U} and \hat{p}_{L}, respectively. The variance (Var) may be the sum of three terms, Var(\hat{p}_{U}−\hat{p}_{L})=V_{S}=V_{C}+V_{M}. The sampling variance V_{S} may represent the unavoidable error in estimating the population frequency from a finite sample. The concentration variance V_{C} may arise from sample-to-sample concentration variations in any one individual's DNA within the pool. The measurement variance may be V_{M}=2ε², where ε is the experimental allele frequency measurement error for each pool. The three sources of variation may be independent, which can be justified when the individual and pooled DNA samples are treated uniformly. In an ideal experiment, V_{C} and V_{M} vanish, and the total variance is from V_{S}.

In a null hypothesis, Z² may have a χ² distribution, preferably, with one degree of freedom under an alternate hypothesis, the tested marker are assumed to be a bi-allelic quantitative trait locus (QTL) with alleles A₁ and A₂ occurring at frequencies p and (1−p)≡q, respectively. According to another aspect of the invention, for between-family tests, the alleles may be assumed to be in Hardy-Weinberg equilibrium and the population may be assumed to have random mating. These assumptions may be relaxed for within-family tests. The preferred variance of the allele frequency per individual is

$${σ}_{p}^{2} = pq / 2.$$

For each design, the allele frequency may be estimated as \hat{p}=(\hat{p}_{U}+\hat{p}_{L})/2. The estimated variance of the allele frequency per individual may be denoted \hat{σ}ₚ² and equals \hat{p}(1−\hat{p})/2.

According to one embodiment of the invention, the mean phenotypic effects may be m_{G}=a, d, and −a for genotypes G=A₁A₁, A₁A₂, and A₂,A₂, respectively. The dominance ratio d/a may describe the inheritance mode with typical values of −1, 0, and 1 for pure recessive, additive, or dominant inheritance. The proportion of trait variance accounted for by the QTL may be denoted

$${σ}_{Q}^{2} ,$$

where

$${σ}_{Q}^{2} = 2 {pq [ a - d ( p - q ) ]}^{2} + {( [ 2 pqd ] )}^{2} = {σ}_{A}^{2} + {σ}_{D}^{2} . \qquad [ 2 ]$$

The mean QTL effect may be m=(p−q)a+2pqd. The phenotypic values may be assumed to be normally distributed for each genotype with a mean μ_{G}=m_{G}−m and a residual variance

$${σ}_{R}^{2} = 1 - {σ}_{Q}^{2}$$

arising from all genetic and environmental factors other than the QTL. The distribution of phenotypic values in the population may be a mixture of the three normal distributions with an overall mean of 0 and a variance of 1. The phenotypic correlation between sibs may be termed t, where t=rh²+σ_{ES}², and where h may represent genetic heritability (including the QTL) and σ_{ES}² may represent shared environmental variance.

According to one embodiment of the invention, a non-centrality parameter (NCP) may be defined as

NCP=[E(\hat{p}_{U}\hat{p}_{L})]²/Var(\hat{p}_{U}−\hat{p}_{L}), [3]

The NCP measures the information provided from a pooled DNA test. In Example 2, the NCP is calculated for between-family and within-family designs.

According to one aspect of the invention, between-family pools may be constructed by ranking the families by mean phenotypic value, then selecting the n₊/s highest families for the upper pool and the n₊/s lowest families (or the lower pool. In one embodiment, the NCP may be the product of three factors, where

$$NCP = \frac{NR {σ}_{1}^{2}}{sT {σ}_{R}^{2}} \cdot \frac{1}{1 + {τ}^{2} / sR} \cdot \frac{2 {y}_{+}^{2}}{{f}_{+} + {f}_{+}^{2} {κ}_{+}^{2}} , \qquad [ 4 ]$$

where

R=(1/s)[1+(s−1)r] [5]

T=(1/s)[σ_{R}²+(s−1)(t−rσ_{A}²−μσ_{D}²)]≈(1/s)[1α(s−1)], [6]

and

$${κ}_{+}^{2} = {ϵ}^{2} / [ ( sR + {τ}^{2} ) ( {σ}_{p}^{2} / N ) ] . \qquad [ 7 ]$$

The pooling fraction f₊ may be n₊/1N, and y₊ may be the height of the standard normal probability density for cumulative probability f₊. The term u in the definition of T may be 1 for monozygotic twins, ½ for full sibs, and 0 for half-sibs. The first factor in equation 4 of the NCP may be the information obtained by a regression test of an additive model based on individual genotyping; the second factor may represent the information lost due primarily to concentration variance; and the third factor may represent the information lost due primarily to measurement error. The preferred optimal pooling fraction may depend only on the normalized measurement error κ₊, wherein the ratio of the measurement error to the standard error of an allele frequency may be estimated by individual genotyping of N/s families of size S.

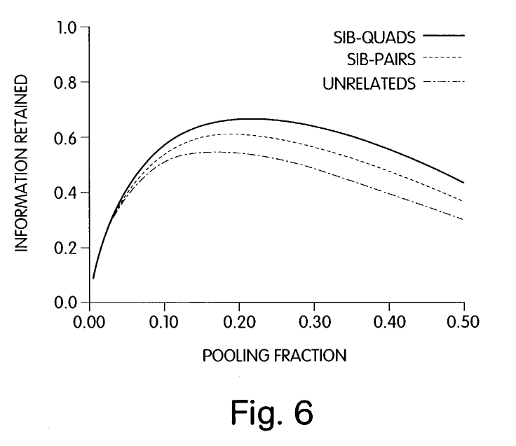

As illustrated in FIG. 6, the information retained by a pooled test, expressed as a fraction of the theoretical maximum from individual genotyping, may be shown as a function of the pooling fraction for three family sizes: sib-quads, sib-pairs, and unrelated individuals.

With increasing family size, sR increases, the information retained increases, and the optimal pooling fraction shifts to higher values. In this example, N=1000 individuals (250, 500, and 1000 families for s=4, 2, and 1, respectively), the allele frequency is p=0.1, there is no concentration variance, and the measurement error is E=0.01. The QTL effect may be assumed to be sufficiently low so that R and T take their limiting values.

According to another aspect of the invention, within-family pools may be constructed by ranking sib-pairs by the difference in phenotypic value, identifying the n₋ sib-pairs with the greatest magnitude difference, then selecting the sib with the higher phenotypic value for the upper pool and the sib with the lower value for the lower pool. In one embodiment, the NCP may be the product of the following three factors,

$$\begin{matrix}NCP = \frac{N ( 1 - R ) {σ}_{A}^{2}}{2 ( 1 - T ) {σ}_{R}^{2}} \cdot \frac{1}{1 + {τ}^{2} / 2 ( 1 - R )} \cdot \frac{2 {y}_{-}^{2}}{{f}_{-} + {f}_{-}^{2} {κ}_{-}^{2}} , \\ with & [ 8 ] \\ {κ}_{-}^{2} = {ϵ}^{2} / \{ [ 2 ( 1 - R ) + {τ}^{2} ] ( {σ}_{p}^{2} / N ) \} . & [ 9 ]\end{matrix}$$

The pooling fraction f₋ may be n₋/N, and the terms R and T may have the same definition as for the between-family pools. The first factor in equation 8 may represent the theoretical maximum information from a regression test of an additive model based on individual genotyping,; the second factor may represent the information lost due primarily to concentration variance; and the third factor may represent the information lost due primarily to measurement error. The normalized measurement error κ₋ may represent the ratio of the measurement error to the standard error of an estimate of (p₁/p₂)/2, which is half the difference in the allele frequency between sibs and with an expectation of 0, from N/2 sib-pairs.

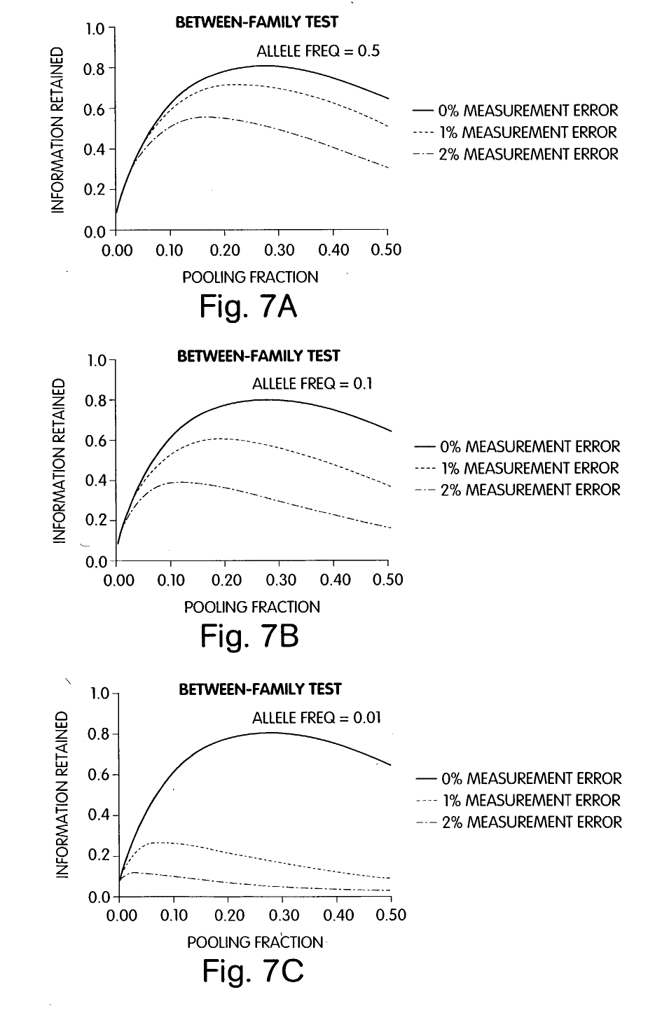

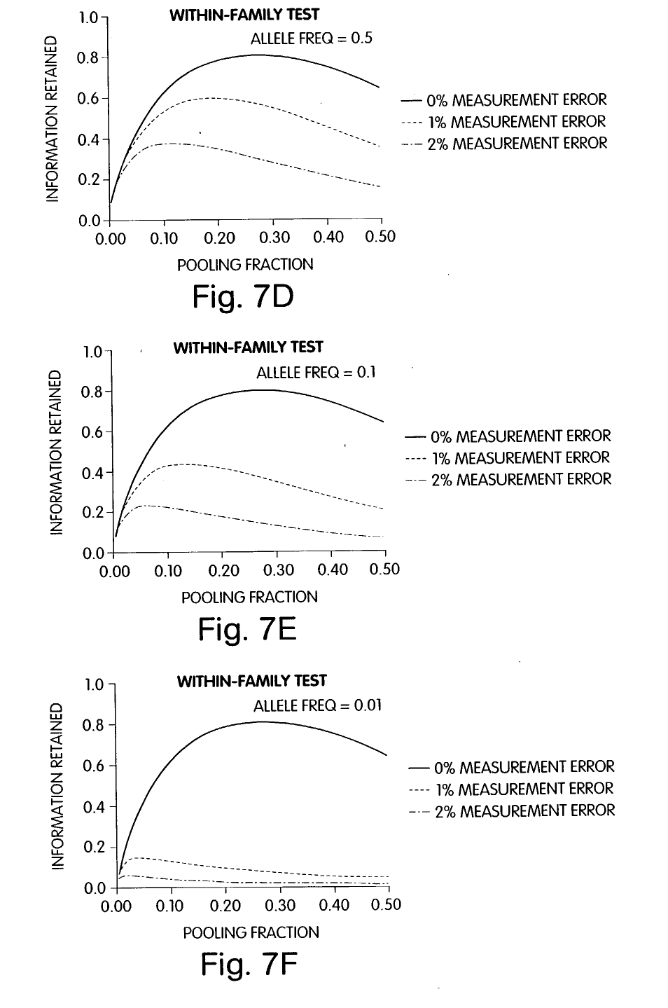

As illustrated in FIG. 7, the information retained may be displayed as a function of the pooling fraction for between-family tests (FIGS. 7A-7C) and within-family tests (FIGS. 7D-7F) for a population of 500 sib-pairs (1000 individuals). The allele frequency may be 0.5 (FIGS. 7A and 7D), 0.1 (FIGS. 7B and 7E), and 0.01 (FIGS. 7C and 7F). For each allele frequency, results may be displayed for measurement errors of 0.0, 0.01, and 0.02. With no measurement error, the optimal pooling fraction of 0.27 will retain 80% of the information in each case. Preferably, as measurement error increases, the optimal pooling fraction decreases, as does the information retained. The information loss may increase for rarer alleles and may be worse for a within-family test than for a between-family test. The concentration variance may be 0 in this example, and the QTL effect may be assumed to be sufficiently small such that R and T take their limiting forms.

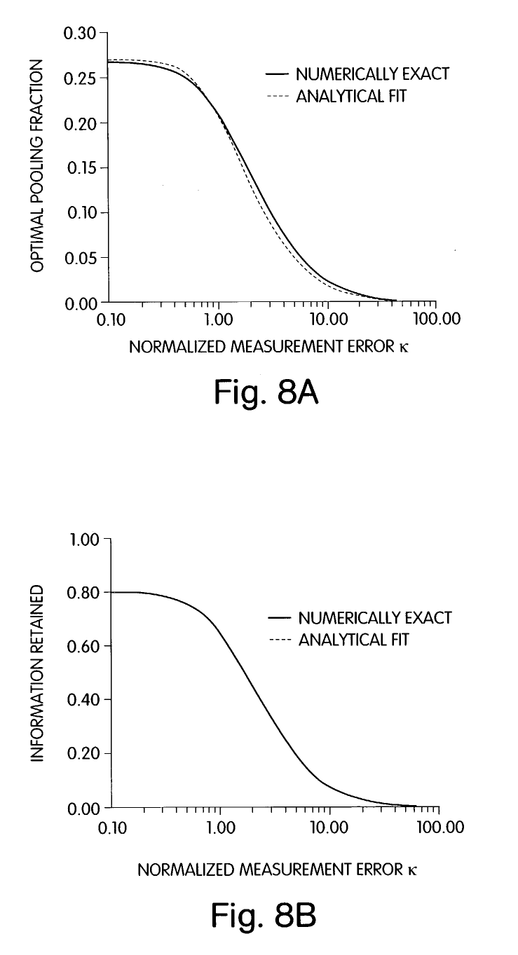

The optimal pooling fraction for each test may depend only on the factor 2y²/(f+/f²κ²). Thus, one can tabulate the optimal fraction as a function of the normalized measurement error κ, can calculate that value of κ that would be appropriate for a particular experiment based on the test design and family structure, the marker frequencies, and the concentration variance and measurement error, then can refer to the table to find the optimal pooling fraction and the information retained. As illustrated in FIG. 8, the optimal pooling fraction (FIG. 8A) and the information retained (FIG. 8B) may be displayed as a function of the normalized measurement error κ. The information retained may be calculated by assuming no concentration variance.

According to one aspect of the invention, in addition to tabulated results, it is preferred to have an analytical fit to the optimal pooling fraction. An accurate fit may be provided by

f=1−Φ[A−(3/A)ln A−0.0067], [10]

where

A(κ)=[2+ln(1+3κ²+2κ⁴/π)]. [11]

The fit is shown as a dashed line in FIG. 8, and a derivation is provided in Example 3. The greatest deviations are at κ=0.5, where the fit yields a pooling fraction that is 0.006 too high, and at κ=3.5, where the fit is 0.01 too low. The information retained using the analytical value for the pooling fraction coincides with the numerical results on the scale of the figure.

In another embodiment of the invention, the NCP may equal [z_{α/2}−z_{1-β}]², where a and a may be the type I and type II error rates for a two-sided test of \hat{p}_{U}−\hat{p}_{L} assuming equal variance under the null and alternate hypothesis. When a p-value is specified, maximizing the NCP may correspond to maximizing the test power.

In one aspect of the invention, one or more designs that include between-family analyses, within-family analyses for large families, and within-family analyses for sib-pairs are considered for estimating the association between at least one genotypic locus and a phenotype. The NCP for each design may be maximized. For each decision, the allele frequency may be estimated as \hat{p}=(\hat{p}_{U}+\hat{p}_{L})/2. The variance of the allele frequency per individual may be denoted as

$${\hat{σ}}_{p}^{2}$$

and may equal \hat{p}(131 \hat{p})/2.

In a different embodiment, the between-family design is used to construct pools by ranking the families by mean phenotypic value, then selecting the n/i families with the highest mean value for the upper pool and the n/s families with the lowest mean value for the lower pool. The preferred sampling variance and concentration variance, derived in Example 1, are

$${V}_{S} + {V}_{C} = 2 sR {\hat{σ}}_{p}^{2} / n + 2 {τ}^{2} {\hat{σ}}_{p}^{2} / n , \qquad [ 12 ]$$

where

R=[1+(s−1)r]/s [13]

and wherein the term τ the coefficient of variation for DNA concentration may be equal to the ratio of the standard deviation of the concentration to its mean.

According to an other aspect of the invention, an analytical expression (or the NCP is valid when

$${σ}_{Q}^{2}$$

is small, derived in Example 2. Here, the NCP is the product of at least four factors. For example,

$$NCP = \frac{N {σ}_{1}^{2}}{{σ}_{R}^{2}} \cdot \frac{R}{sT} \cdot \frac{1}{1 + {τ}^{2} / sR} \cdot \frac{2 {y}^{2}}{f + {f}^{2} {κ}^{2}} , \qquad [ 14 ]$$

where

$$T = ( 1 / s ) [ {σ}_{R}^{2} + ( s - 1 ) ( t - r {σ}_{A}^{2} - u {σ}_{D}^{2} ) ] \approx ( 1 / s ) [ 1 + ( s - 1 ) t ] \qquad [ 15 ]$$

and

$${κ}^{2} = \frac{{ϵ}^{2}}{( sR + {τ}^{2} ) {σ}_{p}^{2} / N} . \qquad [ 16 ]$$

The pooling fraction f may be n/N, and y may be the height of the standard normal probability density for cumulative probability f. The term u in the definition of T is 1 for monozygotic twins, ½ for full sibs, and 0 for half-sibs. The first factor of the ACP in equation 14 may be the information obtained by a regression test of an additive model based on the individual genotyping of an unrelated population; the second factor may be the correction for family structure; the third factor may represent the information lost due primarily to concentration variance; and the fourth factor may represent the information lost due primarily to measurement error. The optimal pooling fraction may depend only on the normalized measurement error κ, preferably the ratio of the measurement error to the standard error of an allele frequency estimated by individual genotyping of N/s families of size v.

As illustrated in FIG. 2, the pooled tests for identifying QTLs may be effectively used in a two-stage design scheme. The sample sizes required for an effective study based on a two-stage design (pooled DNA tests follows by individual genotyping) may need to be calculated first. For example, to perform a genome scan using 100,000 markers, each having a population frequency of 5% or greater, and with a 80% power to identify QTLs responsible for 2% or more of the overall trait variance

$$( {σ}_{A}^{2} / {σ}_{R}^{2} = 0.02 ) ;$$

we may assume access to a homogeneous population and may allow for one (1) false-positive finding. Using the relationship χ²=(z_{α/2}−z_{1-β})² between the expected χ² value, the significance level α/2 for a two-sided test with α=10⁻⁵, the power 1−β=0.8, and the definition z_{α}=Φ⁻¹ (1−α), the critical χ² value may be 27.7. Combining this with the expectation

$${χ}^{2} = N {σ}_{4}^{2} / {σ}_{R}^{2} ,$$

a test based on individual genotyping would indicate that 1360 individuals may be required.

Assuming an assay cost of $0.10, much lower than most current technologies can offer, the total cost may be around $13.6 million.

According to one embodiment of the invention, the best performance obtainable by pooling may be the smallest N satisfying the equation

$$\frac{N {σ}_{A}^{2}}{{σ}_{R}^{2}} \cdot \frac{2 {φ [ {Φ}^{- 1} ( 1 - f ) ]}^{2}}{f + {f}^{2} [ 2 N {ϵ}^{2} / p ( 1 - p ) ]} = {χ}^{2} \geq 27.7 , \qquad [ 17 ]$$

where allele frequencies may be compared between the highest and lowest fN individuals. For the parameters described above and an ε=1% random experimental error, a population of 9500 individuals may be required. The top and bottom 4.1% (390 individuals) may be pooled, retaining 14% of the information in the 9500 individual sample.

At some point, the cost of enrolling a greater number of individuals in a pooling study due to the lower efficiency of pooling, outweighs the benefit of having to perform fewer assays. One possible solution may be to minimize the total cost of a study, including the patient enrolment cost, using a two-stage design in which candidate associations indicated by the pooling are then confirmed by individual genotyping.

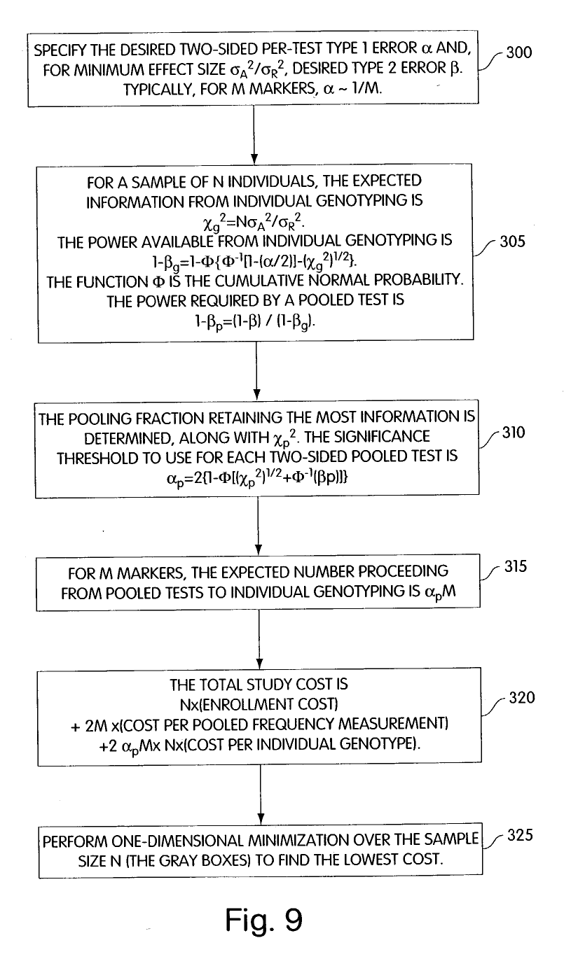

A flow-chart for designing a two-stage study is illustrated in FIG. 9. This flow-chart may be used to minimize the overall cost of a study based on the number of markers, the Type 1 and Type 2 error rates, the random error F in the pooled measurements, the costs of patient enrollment, the pooled allele frequency measurements, and the individual genotyping. The assay development cost may be ignored, assuming cost-sharing over a consortium. As shown in box 300 of FIG. 9, the user specifies the desired two-sided per-test Type 1 error α and, for minimum effect size α_{A}²/σ_{R}²Y, the desired Type 2 error P. Typically, for M markers, α˜1/M may be specified. As shown in box 305, for a sample of N individuals, the expected information from individual genotyping may be χ_{g}²=Nσ_{A}²/σ_{R}².

The power available from individual genotyping may be

1−β_{g}1−Φ{Φ⁻¹[1−(α/2)]−(χ_{g}²)^{1/2}}. [18]

The function Φ may be the cumulative normal probability. The power required by a pooled test may be 1−βₚ=(1−β)/(1−ρ_{g}). As shown in box 310, the pooling fraction retaining the most information may be determined, along with χₚ². The significance threshold to use for each two-sided pooled test may be αₚ=2{1−Φ[(χₚ²)^{1/2}+Φ⁻¹(βₚ]}. As shown in box 315, for M markers, the expected number proceeding from the pooled tests to the individual genotyping may be αₚM. As shown in box 320, the total study cost may be N×(enrollment cost)+2M×(cost per pooled frequency measurement)+2αₚM×N×(cost per individual genotype). As shown in box 325, a one-dimensional minimization may be performed over the sample size N to find the lowest cost.

The least expensive two-phase study, based on an enrollment cost of $1000, a pooled measurement cost of $2, and a $0.50 cost per individual genotype, would require access to 2000 individuals at a total cost of $2.9 million of which $2 million is the enrollment cost. Pooled tests of the present invention can be run on the upper and lower 10% of the population at a cost of $0.4 million using a two-sided significance level of 0.0054, corresponding to 82% power, and yielding approximately 540 false-positive candidates in addition to any true QTLs. Finally, the 540 candidate markers may be genotyped against the entire population at a cost of $0.54 million. Additional savings could be had by genotyping only the individuals with extreme phenotypic values.

3. References

Abecasis G R, Noguchi E, Heinzmann A, Traherne J A, Bhattacharyya A, leaves N I, Anderson G G, Zhang Y, Lench N J, Carey A, Cardon L R, Moffatt M F, Cookson O C (2001) Extent and distribution of linkage disequilibrium in three genomic regions. Am J Hum Gen 68:191-197

Ardlie K G, Kruglyak L, Seielstad M (2002) Patterns of linkage disequilibrium in the human genome. Nat Rev Genet 3: 299-309

Bader J S, Bansal A, and Sham P (2001) Eflicient SNP-based tests of association for quantitative phenotypes using pooled DNA. Genescreen (in press)

Barcellos L F, Klitz W, Field L L, Tobias R, Bowcock A M, Wilson R, Nelson M P, Nagatomi J, Thomson G (1997) Association mapping of disease loci, by use of a pooled DNA genomic screen. Am J. Hum Gen 61:734-747

Collins F S, Guyer M S, Chakarvarti A (1997) Variations on a theme: cataloging human DNA sequence variation. Science 274:1580-1581

Daniels J, Holmans P, Williams N, Turic D, McGuffin P, Plomin R, Owen M J (1998) A simple method for analysing microsatellite allele image patterns generated from DNA pools and its applications to allelic association studies. American Journal of Human Genetics 62:1189-97

Devlin B, Roeder K (1999) Genomic control for association studies. Biometrics 55:788-808

Fisher P J, Turic D, Williams N M, McGuffin P, Asherson P, Ball D, Craig I, Eley T, Hill L, Chorney K, Chorney M J, Benbow C P, Lubiniski D, Plomin R, Owen M J (1999) DNA pooling identifies QTLs on chromosome 4 for general cognitive ability in children. Hum Mol Gen 8: 915-22

Hill L, Craig I W, Asherson P, Ball D, Eley T, Ninomiya T, Fisher P J, Turic D, McGuffin P, Owen M J, Chorney K, Chorney M J, Benbow C P, Lubinski D, Thompson L A, Plomin R (1999) DNA pooling and dense marker maps: a systematic search for genes for cognitive ability. Neuroreport 10: 843-848

Jawaid A, Bader J S, Purcell S, Cherny S S, Sham P (2002) Optimal selection strategies for QTL mapping using pooled DNA samples. European Journal of Human Genetics (in press)

Oft J (1999) Analysis of Human Genetic Linkage. Third edition. Johns Hopkins University Press, Baltimore

Pritchard J K, Stephens M, Rosenberg N A, Donnelly P (2000) Inference of population structure using multilocus genotype data. Genetics 155: 945-959

Pritchard J K, Rosenberg N A (1999) Use of unlinked genetic markers to detect population stratification in association studies. Am J Hum Gen 65: 220-228

Reich D E, Cargill M, Bolk S, Ireland J, Sabeti P C, Richter D J, Lavery T, Kouyoumjiani R, Farhadian S F, Ward R, Lander E S (2001) Linkage disequilibrium in the human genome. Nature 411:199-204

Risch N and Teng J (1998) The relative power of family-based and case-control designs for linkage diequilibrium studies of complex human diseases 1. DNA pooling. Genome Res 8:1273

Risch N, Merikangas K (1996) The future of genetic studies of Complex human diseases. Science 273: 1516-1517

Shaw S H, Carrasquillo M M, Kashuk C, Puffenberger E G, Chakravarti A (1998) Allele frequency distributions in pooled DNA samples: applications to mapping complex disease genes. Genome Res 8: 111-123

Stockton D W, Lewis R A, Abboud E B, A I Rajhi A, Jabak M, Anderson K L, Lupski J R (1998) A novel locus for Leber congenital amaurosis on chromosome 14q24. Human Genetics 103: 328-333

Suzuki K, Bustos T, Spritz R A (1998) Linkage disequilibrium mapping of the gene for Margarita Island ectodermal dysplasia (EZD4) to 11 q23. American Journal of Human Genetics 63:1102-1107

Zhanig S, Zhao H (2001) Quantitative similarity-based association tests using population samples. American Journal of Human Genetics 69: 601-614

### EXAMPLES

### Example 1

### Sampling Variance and Concentration Variance

Let pᵢ represent the frequency of allele A₁ for individual i, such that pᵢ is either 0, ½, or 1, and cᵢ represent the concentration of DNA contributed by this individual to a pool of n individuals. Neglecting measurement error, the allele frequency p* for the pool is

$${p}^{*} = {\sum }_{i} {c}_{i} {p}_{i} / {\sum }_{i} {c}_{i} . \qquad [ 19 ]$$

We assume that cᵢ˜N(c₀,σ_{c}²) and define the coefficient of variation σ_{c}/ρ as τ, with τ much smaller than 1. Expressing cᵢ as c₀+δc₁, with δc₁˜N(0,σ_{c}²), yields

$${p}^{*} = {\sum }_{i} {c}_{i}^{′} {p}_{i} , \qquad [ 20 ]$$

where cᵢ′ is

$${c}_{1}^{′} = [ ( 1 / n ) + ( 1 / n ) ( δ {c}_{i} / {c}_{0} ) ] / [ 1 + ( 1 / n ) {\sum }_{i} ( δ {c}_{i} / {c}_{0} ) ] . \qquad [ 21 ]$$

The root-mean-square magnitude of the second term in the denominator, τ/√n, is much smaller than 1, permitting the expansion (1+δ)⁻¹≈1−δ valid for small δ. This expansion yields

$$\begin{matrix}{c}_{i}^{′} = ( 1 / n ) + ( 1 / n ) ( δ {c}_{i} / μ ) - \\ ( 1 / {n}^{2} ) {\sum }_{j} ( δ {c}_{j} / μ ≡ ( 1 / n ) + δ {c}_{i}^{′} ,\end{matrix} \qquad [ 22 ]$$

which is correct through order 1/n² and δc₁. With this definition,

$$\begin{matrix}E ( δ {c}_{i}^{′} ) = 0 ; & [ 23 ] \\ {\sum }_{i} δ {c}_{i}^{′} = 0 ; and & [ 24 ] \\ Cov ( δ {c}_{i}^{′} , δ {c}_{i}^{′} ) = ( {τ}^{2} / {n}^{2} ) {δ}_{ij} - ( {τ}^{2} / {n}^{3} ) , & [ 25 ]\end{matrix}$$

where δᵢⱼ is 1 if i=j and 0 otherwise.

The allele frequency in the pool may be rewritten

$${p}^{*} = p + ( 1 / n ) {\sum }_{i} δ {p}_{i} + {\sum }_{i} δ {c}_{i}^{′} δ {p}_{i} , \qquad [ 26 ]$$

where δₚ₁ is p−pᵢ. The terms δp₁ and δ_{c}₁′ are uLncoielated, and the variance of p* is

$$\begin{matrix}Var ( {p}^{*} ) = ( 1 / {n}^{2} ) {\sum }_{ij} Cov ( δ {p}_{i} , δ {p}_{j} ) + \\ {\sum }_{i , j}^{Cov} ( δ {c}_{i}^{′} , δ {c}_{j}^{′} ) Cov ( δ {p}_{i} , δ {p}_{j} ) .\end{matrix} \qquad [ 27 ]$$

If the n individuals comprise n/s sib-ships of size s and genotypic correlation r, the result for Var(p*) is

$$Var ( {p}^{*} ) = [ 1 - {τ}^{2} / n ] \cdot [ 1 + ( s - 1 ) r ] {\hat{σ}}_{p}^{2} / n + {τ}^{2} {\hat{σ}}_{p}^{2} / n , \qquad [ 28 ]$$

where the variance of δp₁, \hat{p}(1−\hat{p})/2, is denoted

$${\hat{σ}}_{p}^{2} .$$

Since τ/n is much smaller than 1, the variance may be simplified to read

$$Var ( {p}^{*} ) = [ 1 + ( s - 1 ) r ] {\hat{σ}}_{p}^{2} / n + {τ}^{2} {\hat{σ}}_{p}^{2} / n , \qquad [ 29 ]$$

with the first term identified with the sampling variance V_{S} and the second with the concentration variance V_{C} for a particular pool. For between-family designs, or for unrelated populations, the variances of the two pools may be added to give the final V_{S} and V_{C}.

For the within-family design for sib pairs, the allele frequency difference between pools is

$$Δ {p}^{*} = ( 1 / n ) {\sum }_{k} ( δ {p}_{k1} - δ {p}_{k2} ) + {\sum }_{k} δ {c}_{k1}^{′} δ {p}_{k1} - {\sum }_{k} δ {c}_{k2}^{′} δ {p}_{k2} , \qquad [ 30 ]$$

The index k denotes the family; within each family, sib 1 is selected for the upper pool and sib 2 is selected for the lower pool. Each of the three terms on the right hand side is uncorrelated from the other two and contributes additively to the total variance. The latter two terms, each with variance

$${τ}^{2} {\hat{σ}}_{p}^{2} / n ,$$

are identified with V_{C}. The variance of the first term is V_{S}. When 2n/s families of size s are identified and the sibs are split evenly between pools, V_{S} may be written

$$\begin{matrix}{V}_{S} = ( 1 / {n}^{2} ) {\sum }_{k} \{ {\sum }_{{ii}^{′}} Cov ( δ {p}_{ki} , δ {p}_{{ki}^{′}} ) + \\ {\sum }_{{ii}^{′}} Cov ( δ {p}_{j} , δ {p}_{{kj}^{′}} ) - \\ 2 {\sum }_{ij} Cov ( δ {p}_{ki} , δ {p}_{kj} ) \} ,\end{matrix} \qquad [ 31 ]$$

where, for each family, i and i′ designate the s/2 sibs selected for the upper pool, and j and j′ designate the s/2 sibs selected for the lower pool. Performing the sums yields

$$\begin{matrix}{V}_{S} = ( 4 {\hat{σ}}_{p}^{2} / n s ) \{ ( s / 2 ) [ 1 + s / 2 - 1 ) r ] - \\ {( s / 2 )}^{2} r \} \\ = 2 ( 1 - r ) {\hat{σ}}_{p}^{2} / n .\end{matrix} \qquad [ 32 ]$$

The result is independent of s.

### Example 2

### Expected Allele Frequency Difference

Defining the terms in a standard variance components model,

Xₖᵢ=Yₖ+Yₖᵢ+μ(Gₖᵢ), [33]

$$\begin{matrix}{Y}_{k} ∼ N ( 0 , t - r {σ}_{A}^{2} - u {σ}_{D}^{2} ) , & [ 34 ] \\ {Y}_{ki} ∼ N ( 0 , {σ}_{R}^{2} - t + r {σ}_{Q}^{2} + u {σ}_{D}^{2} ) , & [ 35 ]\end{matrix}$$

where Xₖᵢ is the phenotypic value of sib i from family k, Yₖ represents the sib-ship shared effect excluding the QTL, Yₖᵢ represents the individual non-shared effect excluding the QTL, and μ(Gₖᵢ) is the mean effect from the QTL and depends on the genotype Gₖᵢ of the sib. The genotypic correlation between sibs is r, and it u is 1 for monozygotic twins, ¼ for full sibs, and 0 for half sibs.

For a between-family design, let X_{k•} represent the average of the individual phenotypic values for family k with s sibs,

$$\begin{matrix}{X}_{k •} = ( 1 / s ) {\sum }_{j = 1}^{i} {X}_{kj} = {Y}_{k •} + {μ}_{k •} , & [ 36 ] \\ \begin{matrix}{Y}_{k •} ∼ N ( 0 , ( 1 / s ) [ {σ}_{R}^{2} + ( s - 1 ) ( t - r {σ}_{1}^{2} - u {σ}_{D}^{2} ) ] ) \\ = N ( 0 , T {σ}_{R}^{2} ) ,\end{matrix} \\ and & [ 37 ] \\ {μ}_{k •} = ( 1 / s ) {\sum }_{i} μ ( {G}_{ki} ) . & [ 38 ]\end{matrix}$$

The second equation serves to define the term T, which has the limit[1+(s−1)t]/s when the QTL effect approaches 0.

Suppose the n/s families with greatest family average X_{k•} are selected for a pool of n individuals. Using f to represent the pooling fraction n/N,

$$f = {\sum }_{G} P ( G ) {\int }_{{X}_{U}}^{∞} ⅆ {X ( 2 π T {σ}_{R}^{2} )}^{- 1 / 2} exp [ - {( X - {μ}_{G} )}^{2} / 2 T {σ}_{R}^{2} ] , \qquad [ 39 ]$$

where G represents the genotypes G₁, G₂, . . . , Gₛ for a sib-ship of sizes, P(G) is the corresponding joint probability distribution normalized to 1, and μ_{G} is the QTL effect for a family corresponding to the term μ_{k•} in the variance components model. The mean of u_{G}, Σ_{G}P(G)μ_{G}, is 0. 25

While the equation for f may be inverted numerically to obtain the pooling threshold X_{U} as a function of the model parameters, an analytical approximation valid in the limit of small QTL effect may be obtained by expanding the exponential and keeping terms through order m_{G},

$$\begin{matrix}f = {\sum }_{G} P ( G ) {\int }_{{X}_{b}}^{∞} ⅆ {X ( 2 π T {σ}_{R}^{2} )}^{- 1 / 2} ( 1 + {μ}_{G} X / T {σ}_{R}^{2} ) \\ exp [ - {X}^{2} / 2 T {σ}_{R}^{2} ] \\ = Φ ( - {X}_{U} / {T}^{1 / 2} {σ}_{R} ) ,\end{matrix} \qquad [ 40 ]$$

where Φ(z) is the cumulative probability distribution for standard normal deviate z. Inverting this equation yields −T^{1/2}σ_{R}Φ⁻¹(f) as the pooling threshold, where Φ⁻¹ (f) is the inverse cumulative standard normal probability distribution.

The expected allele frequency for the upper pool, E(\hat{p}_{U}), is obtained as

$$\begin{matrix}E ( {\hat{p}}_{U} ) = ( 1 / f ) {\sum }_{G} P ( G ) {p}_{G} {\int }_{{X}_{L}}^{∞} ⅆ {X ( 2 π T {σ}_{R}^{2} )}^{- 1 / 2} \\ exp [ - {( X - {μ}_{G} )}^{2} / 2 T {σ}_{R}^{2} ] , \end{matrix} \qquad [ 41 ]$$

where p_{G} is the average allele frequency for a sib-ship with genotypes G,

$${p}_{G} = ( 1 / s ) {\sum }_{i = 1}^{i} p ( {G}_{i} ) , \qquad [ 42 ]$$

and p(G) is 0, ½, or 1 depending on genotype G. The expectation E(\hat{p}_{U}) may be obtained numerically using the numerical solution for f. Alternatively, for small QTL effect, an analytical approximation may be obtained by expanding the exponential through terms of order m_{G},

$$\begin{matrix}E ( {\hat{p}}_{U} ) = ( 1 / f ) {\sum }_{G} P ( G ) {p}_{G} {\int }_{{X}_{i}}^{∞} ⅆ {X ( 2 π T {σ}_{R}^{2} )}^{- 1 / 2} \\ ( 1 + {μ}_{G} X / T {σ}_{R}^{2} ) exp [ - {X}^{2} / 2 T {σ}_{R}^{2} ] .\end{matrix} \qquad [ 43 ]$$

Inserting the analytical expression for X_{U} and performing the integrals over X yields

$$E ( {\hat{p}}_{U} ) = p + ( y / {fT}^{1 / 2} {σ}_{R} ) {\sum }_{G} P ( G ) {p}_{G} {μ}_{G} , \qquad [ 44 ]$$

where y is the standard normal probability density (2π)^{1/2} exp {−[Φ⁻¹(f)]²/2} corresponding to cumulative probability f.

Because p_{G} and μ_{G} are both linear in sib variables, the mean of p_{G}μ_{G} can be obtained by considering pair-wise correlations p(Gᵢ)μ(Gⱼ) for a particular pair of sibs i and i with genotypes Gᵢ and Gⱼ Since p(Gᵢ) projects the additive component of the QTL effect, the mean of p(Gᵢ)μ(Gⱼ) is rᵢⱼE[p(G)μ(G)], where i, is the genotypic correlation between sibs i and j. (This result may be confirmed by an explicit calculation using a table of sib-pair genotype probabilities for full-sibs or half-sibs.) The expectation for an individual is

$$\begin{matrix}E [ p ( G ) μ ( G ) ] = {\sum }_{G = {A}_{1} {A}_{1} , {A}_{1} {A}_{2} , {A}_{2} {I}_{2}} P ( G ) p ( G ) μ ( G ) \\ = pq [ a - ( p - q ) d ] \\ = {σ}_{P} {σ}_{A} ,\end{matrix} \qquad [ 45 ]$$

and the corresponding result for a family is

$$\begin{matrix}{\sum }_{G} P ( G ) {p}_{G} {μ}_{G} = {\sum }_{G} P ( G ) ( 1 / {s}^{2} ) {\sum }_{i = 1} p ( {G}_{i} ) m ( {G}_{j} ) \\ = ( 1 / s ) [ 1 + ( s - 1 ) r ] {σ}_{p} {σ}_{A} ≡ R {σ}_{p} {σ}_{A} ,\end{matrix} \qquad [ 46 ]$$

where r is the genotypic correlation for each pair of sibs. This equation also serves to define the term R.

The expected allelc frequency for the upper pool is

E(\hat{p}_{U})=p+(yR/fT^{1/2})(σₚσ₄/σ_{R}). [47]

By symmetry, the lower pool has an offset of equal magnitude and opposite direction, yielding an expected allele frequency difference of

$$E ( {\hat{p}}_{U} - {\hat{p}}_{L} ) = 2 yR {σ}_{p} \frac{{σ}_{A}}{{fT}^{1 / 2} {σ}_{R}} \qquad [ 48 ]$$

when the QTL effect is small.

Recalling the terms contribute in to the variance of the estimator,

V_{S}=2sRσₚ²/fN [49]

and

V_{C}=2τ²σₚ²/fN [50]

the NCP for the between-family design is obtained as

$$\begin{matrix}NCP = \frac{NR {σ}_{A}^{2}}{sT {σ}_{R}^{2}} \cdot \frac{1}{1 + {τ}^{2} / sR} \cdot \frac{2 {y}_{+}^{2}}{{f}_{+} + {f}_{+}^{2} {κ}_{+}^{2}} , with & [ 51 ] \\ {κ}^{2} = {ϵ}^{2} / [ ( sR + {τ}^{2} ) ( {σ}_{P}^{2} / N ) ] . & [ 52 ]\end{matrix}$$

For the within-family pool design, we restrict attention to sib-pairs. For each family k, half the phenotype difference between sibs 1 and 2 is denoted ΔXₖ=(Xₖ₁−Xₖ₂)/2. In terms of the variance components model,

ΔXₖ=ΔYₖ+Δμₖ, [53]

where

$$Δ {Y}_{k} \sim N [ 0 , ( {σ}_{R}^{2} - t + r {σ}_{A}^{2} + u {σ}_{D}^{2} ) / 2 ] = N [ 0 , ( 1 - T ) {σ}_{R}^{2} ] \qquad [ 54 ]$$

and

Δμₖ=[μ(Gₖ₁)−μ(Gₖ₂)]/2 [55]

The definition of T in the middle equation is identical to that for the between-family design with s=2. Families are ranked by |ΔXₖ|, and the n families having the largest magnitude are identified as the source of the 2n individuals to be pooled. The threshold magnitude is denoted X₁ and is related to the pooling fraction f through the following equation.

$$f = ( 1 / 2 ) {\sum }_{G} P ( G ) [ {\int }_{- ∞}^{- {A}_{i}} + {\int }_{{V}_{i}}^{∞} ] ⅆ {X [ 2 π ( 1 - T ) {σ}_{R}^{2} ]}^{- 1 / 2} exp [ - {( X - Δ {μ}_{G} )}^{2} / 2 ( 1 - T ) {σ}_{R}^{2} ] \qquad [ 56 ]$$

The leading factor of (½) indicates that only 1 sib is selected for each pool, and the term Δμ_{G} corresponds to the term Δμₖ in the variance components model for (G=(G₁,G₂).

While it is possible to invert this equation numerically to obtain X_{T} as a function of f, an analytical approximation derived by expanding the exponential to lowest order in Δμ_{G},

$$exp [ - {( X - {Δμ}_{G} )}^{2} / 2 ( 1 - T ) {σ}_{R}^{2} ] \approx [ 1 + X {Δμ}_{G} / ( 1 - T ) {σ}_{R}^{2} ] exp [ - {X}^{2} / 2 ( 1 - T ) {σ}_{R}^{2} ] \qquad [ 57 ]$$

is very accurate for QTLs with small effect. The result for the pooling fraction is

f=Φ[−X₁/(1−T)^{1/2}σ_{R}]. [58]

The expected allele frequency difference between pools is

$$E ( {\hat{p}}_{U} - {\hat{p}}_{L} ) = ( 1 / 2 ) f {\sum }_{G} P ( G ) [ p ( {G}_{1} ) - p ( {G}_{2} ) ] \times [ - {\int }_{- ∞}^{- {V}_{i}} + {\int }_{{V}_{i}}^{∞} ] ⅆ {X [ 2 π ( 1 - T ) {σ}_{R}^{2} ]}^{- 1 / 2} exp [ - {( X - Δ {μ}_{G} )}^{2} / 2 ( 1 - T ) {σ}_{R}^{2} ] \qquad [ 59 ]$$

and may be calculated numerically. Alternately, the low-order expansion for the exponential may be inserted to yield

$$E ( {\hat{p}}_{U} - {\hat{p}}_{L} ) = ( 1 / 2 f ) {\sum }_{G} P ( G ) [ p ( {G}_{1} ) - p ( {G}_{2} ) ] \cdot 2 y {Δμ}_{G} / {( 1 - T )}^{1 / 2} {σ}_{R} , \qquad [ 60 ]$$

probability is f.

The genotype-dependent sum is

$$\begin{matrix}{\sum }_{G} P ( G ) [ p ( {G}_{1} ) - p ( {G}_{2} ) ] {Δμ}_{G} = ( 1 / 2 ) {\sum }_{G} P ( G ) \{ p ( {G}_{1} ) μ ( {G}_{1} ) + \\ p ( {G}_{2} ) μ ( {G}_{2} ) - p ( {G}_{1} ) μ ( {G}_{2} ) - \\ p ( {G}_{2} ) μ ( {G}_{1} ) \} \\ = ( 1 - r ) {σ}_{p} {σ}_{A} = 2 ( 1 - R ) {σ}_{p} {σ}_{A}\end{matrix} \qquad [ 61 ]$$

where R has the same definition as for the between-family design. Inserting this into the previous equation yields

$$E ( {\hat{p}}_{U} - {\hat{p}}_{L} ) = 2 y ( 1 - R ) {σ}_{p} \frac{{σ}_{A}}{{f ( 1 - T )}^{1 / 2} {σ}_{R}} \qquad [ 62 ]$$

for the expected allele frequency difference. Recalling the variance of the estimator,

$$Var ( {\hat{p}}_{U} - {\hat{p}}_{L} ) = 4 ( 1 - R ) {σ}_{p}^{2} / Nf + 2 {τ}^{2} {σ}_{p}^{2} / Nf + 2 {ϵ}^{2} \qquad [ 63 ]$$

yields for the NCP the value

$$\begin{matrix}NCP = \frac{{N ( 1 - R )}^{2} {σ}_{A}^{2}}{( 1 - T ) [ 2 ( 1 - R ) + {τ}^{2} ] {σ}_{R}^{2}} \cdot \frac{2 {y}^{2}}{f + {f}^{2} {κ}^{2}} , with & [ 64 ] \\ {κ}^{2} = {ϵ}^{2} / \{ [ 2 ( 1 - R ) + {τ}^{2} ] ( {σ}_{p}^{2} / N ) \} . & [ 65 ]\end{matrix}$$

### Example 3

### Analytical Fit for the Optimal Pooling Fraction

The pooling fraction is optimized to maximize the value of the information retained by the NCP, which is equivalent to maximizing the value of

1=2y²/(f+f²κ²). [66]

Both y and f may be expressed in terms of a normal deviate z,

y=exp(−z²/2)/\sqrt{2π}, [67]

and

f=Φ(−Z), [68]

where the use of −z in the definition or f provides z>0 for convenience. Taking the derivative of 1 with respect to z and dividing by non-zero terms,

y·(1+2fκ²)−2zf·(1+fκ²)=0 [69]

yields the optimum; we have used dy/dz=−yz and df/dz=−y.

When κ² is large, z is also large, and f may be replaced by its asymptotic expansion for large z,

f=y·(z⁻¹−z⁻³). [70]

With this substitution, the optimum satisfies

z³/2yκ²=1 [71]

Taking the natural logarithm of both sides and equating exponents,

J(z)=z²/2+3 ln z−ln(κ²\sqrt{2/π}). [72]

When κ and z are both large, the term proportional to ln z is asymptotically small, and the asymptotic result for z is

z˜B(κ)≡\sqrt{ln(2κ⁴π)}. [73]

An improved fit is obtained by perturbation theory by writing

z=B(κ)[1+b(κ)], [74]

where

$${lim}_{A \to ∞} b ( κ ) = 0.$$

Substituting this expression for z into J(z) and simplifying,

B²b+3ln [B(1+b)]=0, [75]

which gives the asymptotic form

b=(3/B²)ln B, [76]

or

z˜B−(3/B)ln B. [77]

This form provides a good fit when κ is much larger than 1, but not for smaller values. Since the asymptotic behavior for large κ is not affected by introducing terms of lower order in κ, the fit can he improved for small κ without affecting the fit at large κ by writing

z=A−(3/A)ln A+a₁, [78]

where

A(κ)=\sqrt{a₂+ln(1+a₃κ²+2κ⁴π)}. [79]

The constants a₁, a₂, and a₃ are then selected to fit the exact numerical results at particular-values of κ. Fitting the results z=0.612 at κ=0 and z=0.8047 at κ=1 provides the particular parameters

a₁=−0.067, a₂=2, a₃=3. [80]

### Example 4

### Between-Family Sampling Variance and Concentration Variance

Let pᵢ represent the frequency of allele A₁ for individual i, such that pᵢ is either 0, ½, or 1, and cᵢ represent the concentration of DNA contributed by this individual to a pool of n individuals. Neglecting measurement error, the allele frequency p* for the pool is

$${p}^{*} = {\sum }_{i } {c}_{i} {p}_{i} / {\sum }_{i} {c}_{i} . \qquad [ 81 ]$$

We assume that cᵢ˜N(c₀,σ_{c}²) and define the coefficient of variation σ_{c}/μ as τ, with τ much smaller than 1. Expressing cᵢ as c₀+δ_{c}₁, with δ_{c}₁˜N(0,σ_{c}²), yields

$$\begin{matrix}{p}^{*} = {\sum }_{i} {c}_{i}^{′} {p}_{i} , \\ where {c}_{i}^{′} is & [ 82 ] \\ {c}_{i}^{′} = [ ( 1 / n ) + ( 1 / n ) ( δ {c}_{i} / {c}_{0} ) ] / [ 1 + ( 1 / n ) {\sum }_{j} ( δ {c}_{j} / {c}_{0} ) ] . & [ 83 ]\end{matrix}$$

The root-mean-square magnitude of the second term in the denominator, τ/√n, is much smaller than 1, permitting the expansion (1+δ)⁻¹≈1−δ valid for small δ. This expansion yields

$${c}_{i}^{′} = ( 1 / n ) + ( 1 / n ) ( δ {c}_{i} / μ ) - ( 1 / {n}^{2} ) {\sum }_{j} ( δ {c}_{j} / μ ) ≡ ( 1 / n ) + δ {c}_{i}^{′} , \qquad [ 84 ]$$

which is correct through order 1/n² and δc₁. With this definition,

E(δc₁′)=0; [85]

$$\begin{matrix}{\sum }_{i} δ {c}_{i}^{′} = 0 ; and & [ 86 ] \\ Cov ( δ {c}_{i}^{′} , δ {c}_{j}^{′} ) = ( {τ}^{2} / {n}^{2} ) {δ}_{ij} - ( {τ}^{2} / {n}^{3} ) , & [ 87 ]\end{matrix}$$

where δᵢⱼ is 1 if i=j and 0 otherwise. The allele frequency in the pool may be rewritten

$${p}^{*} = p + ( 1 / n ) {\sum }_{i} δ {p}_{i} + {\sum }_{i} δ {c}_{i}^{′} δ {p}_{i} , \qquad [ 88 ]$$

where δpᵢ is pᵢ−p. The terms δp₁ and δcᵢ′ are uncorrelated, and the variance of p is

$$Var ( {p}^{*} ) = ( 1 / {n}^{2} ) {\sum }_{i , j} Cov ( δ {p}_{i} , δ {p}_{j} ) + {\sum }_{i , j} Cov ( δ {c}_{i}^{′} , δ {c}_{j}^{′} ) Cov ( δ {p}_{i} , δ {p}_{j} ) . \qquad [ 89 ]$$

For the between-family design, the n individuals comprise n/s sib-ships of size s and genotypic correlation r, and the result for Var(p*) is

$$Var ( {p}^{*} ) = [ 1 - {τ}^{2} / n ] \cdot [ 1 + ( s - 1 ) r ] {\hat{σ}}_{p}^{2} / n + {τ}^{2} {\hat{σ}}_{p}^{2} / n . \qquad [ 90 ]$$

The variance of δp₁, \hat{p}(1−\hat{p})/2, has been denoted \hat{σ}ₚ². Since τ²/n is much smaller than 1, the variance may be simplified to read

$$Var ( {p}^{*} ) = sR {\hat{σ}}_{p}^{2} / n + {τ}^{2} {\hat{σ}}_{p}^{2} / n , \qquad [ 100 ]$$

with the first term identified with the sampling variance V_{S} and the second with the concentration variance V_{C} for a particular pool. The genotypic correlation is represented by R, defined as

R=[1+(s−1)r]/s. [101]

The variances of the upper and lower pools are added to give the final V_{S} and V_{C},

$${V}_{S} + {V}_{C} = 2 s R {\hat{σ}}_{p}^{2} / n + 2 {τ}^{2} {\hat{σ}}_{p}^{2} / n . \qquad [ 102 ]$$

### Example 5

### Within-Family Sampling Variance and Concentration Variance

For the within-family designs, the allele frequency difference between pools is

$$\begin{matrix}Δ {p}^{*} = ( 1 / n ) {\sum }_{k = 1}^{n / {s}^{′}} {\sum }_{j = 1}^{{s}^{′}} ( δ {p}_{ki} - δ {p}_{kj} ) + \\ {\sum }_{k = 1}^{n / {s}^{′}} {\sum }_{i = 1}^{{s}^{′}} δ {c}_{ki}^{′} δ {p}_{ki} - {\sum }_{k = 1}^{n / {s}^{′}} {\sum }_{j = 1}^{{s}^{′}} δ {c}_{kj}^{′} δ {p}_{kj} .\end{matrix} \qquad [ 103 ]$$

The index k denotes the family, with 2s′ sibs selected from each of n/s′ families. For each family, the index i denotes sibs selected for the upper pool and j denotes sibs selected for the lower pool, with both i and j running from 1 to s′. Each of the three terms on the right hand side is uncorrelated from the other two and contributes additively to the total variance. The latter two terms, each with variance

$$[ {τ}^{2} {σ}_{p}^{2} / n ] \cdot [ 1 - {s}^{′} {R}^{′} / n ] ,$$

are identified with V_{C}, where R′=[1+(s−1)r]/s′. When the pool size n is large, term s′R′/n in V_{C} is much smaller than 1 and may be neglected.

The variance of the first term is V_{S}.

$$\begin{matrix}{V}_{S} \approx ( 1 / {n}^{2} ) \{ {\sum }_{ki} {\sum }_{{k}^{′} {i}^{′}} Cov ( δ {p}_{ki} , δ {p}_{{k}^{′} {j}^{′}} ) + \\ {\sum }_{k , j} {\sum }_{{k}^{′} , {j}^{′}} Cov ( δ {p}_{kj} , δ {p}_{{k}^{′} {j}^{′}} ) - \\ 2 {\sum }_{ki} {\sum }_{{k}^{′} , {i}^{′}} Cov ( δ {p}_{ki} , δ {p}_{{k}^{′} {j}^{′}} ) \} .\end{matrix} \qquad [ 104 ]$$

Performing the sums yields

$${V}_{S} = ( 1 / {n}^{2} ) \{ 2 n {\hat{σ}}_{p}^{2} [ 1 + ( {s}^{′} - 1 ) r ] - 2 n {\hat{σ}}_{p}^{2} {s}^{′} r \} , \qquad [ 105 ]$$

which simplifies to

$${V}_{S} + {V}_{C} = 2 ( 1 - r ) {\hat{σ}}_{p}^{2} / n + 2 {τ}^{2} {\hat{σ}}_{p}^{2} / n . \qquad [ 106 ]$$

### Example 6

### Within-Family Expected Allele Frequency Difference

Defining the terms in a standard variance components model,

Xₖᵢ=Yₖ+Yₖᵢ+μₖᵢ, [107]

$$\begin{matrix}{Y}_{k} ∼ N ( 0 , t - r {σ}_{A}^{2} - u {σ}_{D}^{2} ) , & [ 108 ] \\ {Y}_{ki} ∼ N ( 0 , {σ}_{R}^{2} - t + r {σ}_{A}^{2} + u {σ}_{D}^{2} ) , & [ 109 ]\end{matrix}$$

where Xₖᵢ is the phenotypic value of sib i from family k, Yₖ represents the sib-ship shared effect excluding the QTL, Yₖᵢ represents the individual non-shared effect excluding the QTL, and μₖᵢ is an abbreviation for μ(Gₖᵢ), the QTL effect for sib i. The genotypic correlation between sibs is r, and u is 1 for monozygotic twins, ½ for full sibs, and 0 for half sibs.

For a between-family design, let X_{k•} represent the average of the individual phenotypic values for family k with s sibs,

$$\begin{matrix}{X}_{k•} = ( 1 / s ) {\sum }_{j = 1}^{s} {X}_{kj} = {Y}_{k•} + {μ}_{k•} , & [ 110 ] \\ \begin{matrix}{Y}_{k•} ∼ N ( 0 , ( 1 / s ) [ {σ}_{R}^{2} + ( s - 1 ) ( t - r {σ}_{A}^{2} - {μσ}_{D}^{2} ) ] ) \\ = N ( 0 , T {σ}_{R}^{2} ) ,\end{matrix} \\ and & [ 111 ] \\ {μ}_{k•} = ( 1 / s ) {\sum }_{i} {μ}_{ki} . & [ 112 ]\end{matrix}$$

The second equation serves to define the term T, which has the limit [1+(s−1)t]/s when the QTL, effect approaches 0.

Under the between-family design, the n/s families with greatest family average X_{k•} are selected for a pool of n individuals. Using f to represent the pooling fraction n/N,

$$\begin{matrix}f = {\sum }_{G} P ( G ) {\int }_{{X}_{0}}^{∞} ⅆ {X ( 2 π T {σ}_{R}^{2} )}^{- 1 / 2} \\ exp [ - {( X - {μ}_{G} )}^{2} / 2 T {σ}_{R}^{2} ] ,\end{matrix} \qquad [ 113 ]$$

where G represents the genotypes G₁, G₂, . . . , Gₛ for a sib-ship of size s, P(G) is the corresponding joint probability distribution normalized to 1, and μ_{G} is the QTL effect for a family corresponding to the term μ_{k•} in the variance components model. The mean of μ_{G}, Σ_{G}P(G)μ_{G}, is 0.

While the equation for f may be inverted numerically to obtain the pooling threshold X_{U} as a function of the model parameters, an analytical approximation valid in the limit of small QTL effect may be obtained by expanding the exponential and keeping terms through order μ_{G},

$$\begin{matrix}f = {\sum }_{G} P ( G ) {\int }_{{X}_{t}}^{∞} ⅆ {X ( 2 π T {σ}_{R}^{2} )}^{- 1 / 2} ( 1 + {μ}_{G} X / T {σ}_{R}^{2} ) \\ exp [ - {X}^{2} / 2 T {σ}_{R}^{2} ] \\ = Φ ( - {X}_{U} / {T}^{1 / 2} {σ}_{R} ) ,\end{matrix} \qquad [ 114 ]$$

where Φ(z) is the cumulative probability distribution for standard normal deviate z. Inverting this equation yields −T^{1/2}σ_{R}Φ⁻¹ (f) as the pooling threshold, where Φ⁻¹(f) is the inverse cumulative standard normal probability distribution.

The expected allele frequency for the upper pool, E(\hat{p}_{U}), is obtained as

$$\begin{matrix}E ( {\hat{p}}_{U} ) = ( 1 / f ) {\sum }_{G} P ( G ) {p}_{G} {\int }_{{X}_{t}}^{∞} ⅆ {X ( 2 π T {σ}_{R}^{2} )}^{- 1 / 2} \\ exp [ - {( X - {μ}_{G} )}^{2} / 2 T {σ}_{R}^{2} ] ,\end{matrix} \qquad [ 115 ]$$

where p_{G} is average allele frequency for a sib-ship with genotypes G,

$${p}_{G} = ( 1 / s ) {\sum }_{i = 1}^{s} p ( {G}_{i} ) , \qquad [ 116 ]$$

and p(G) is 0, ½, or 1 depending on genotype G. The expectation E(\hat{p}_{U}) may be obtained numerically using the numerical solution for f. Alternatively, for small QTL effect, an analytical approximation may be obtained by expanding the exponential through terms of order μ_{G},

$$\begin{matrix}E ( {\hat{p}}_{U} ) = ( 1 / f ) {\sum }_{G} P ( G ) {p}_{G} {\int }_{{X}_{L}}^{∞} ⅆ {X ( 2 π T {σ}_{R}^{2} )}^{- 1 / 2} \\ ( 1 + {μ}_{G} X / T {σ}_{R}^{2} ) exp [ - {X}^{2} / 2 T {σ}_{R}^{2} ] .\end{matrix} \qquad [ 117 ]$$

Inserting the analytical expression for X_{U} and performing the integrals over X yields

$$E ( {\hat{p}}_{U} ) = p + ( y / {fT}^{1 / 2} {σ}_{R} ) {\sum }_{G} P ( G ) {p}_{G} {μ}_{G} , \qquad [ 118 ]$$

where y is the standard normal probability density (2π)^{-1/2} exp{−[Φ⁻¹(f)]²/2} corresponding to cumulative probability f.

Because p_{G} and μ_{G} are both linear in sib variables, the mean of p_{G}μ_{G} can be obtained by considering pair-wise correlations p(Gᵢ)μ(Gⱼ) for a particular pair of sibs i and j with genotypes Gᵢ and Gⱼ. Since p(Gᵢ) projects the additive component of the QTL effect, the mean of p(Gᵢ)λ(Gⱼ) is rᵢⱼE[p(G)μ(G)], where rᵢⱼ is the genotypic correlation between sibs i and j. (This result may be confirmed by an explicit calculation using a table of sib-pair genotype probabilities for full-sibs or half-sibs.) The expectation for an individual is

$$\begin{matrix}E [ p ( G ) μ ( G ) ] = {\sum }_{G = {A}_{1} {A}_{1} , {A}_{1} {A}_{2} {A}_{2} {A}_{2} {A}_{2}} P ( G ) p ( G ) μ ( G ) \\ = pq [ a - ( p - q ) d ] \\ = {σ}_{p} {σ}_{A} ,\end{matrix} \qquad [ 119 ]$$

and the corresponding result for a family is

$$\begin{matrix}{\sum }_{G} P ( G ) {p}_{G} {μ}_{G} = {\sum }_{G} P ( G ) ( 1 / {s}^{2} ) {\sum }_{i , j} p ( {G}_{i} ) m ( {G}_{l} ) \\ = ( 1 / s ) [ 1 + ( s - 1 ) r ] {σ}_{p} {σ}_{t} \\ ≡ R {σ}_{p} {σ}_{A} ,\end{matrix} \qquad [ 120 ]$$

where r is the genotypic correlation for each pair of sibs. This equation also serves to define the term R.

The expected allele frequency for the upper pool is

E(\hat{p}_{U})=p+(yR/fT^{1/2})(σₚσ₄/σ_{R}). [121]

By symmetry, the lower pool has an offset of equal magnitude and opposite direction, yielding an expected allele frequency difference of

$$E ( {\hat{p}}_{U} - {\hat{p}}_{i} ) = 2 yR {σ}_{p} \frac{{σ}_{A}}{{fT}^{1 / 2} {σ}_{R}} \qquad [ 122 ]$$

when the QTL effect is small.

Dividing the square of the expected allele frequency difference by its variance gives the NCP for the between-family design,

$$\begin{matrix}NCP = \frac{N {σ}_{1}^{2}}{{σ}_{R}^{2}} \cdot \frac{R}{sT} \cdot \frac{1}{1 + {τ}^{2} / sR} \cdot \frac{2 {y}^{2}}{f + {f}^{2} {κ}^{2}} , \\ with & [ 123 ] \\ {κ}^{2} = \frac{{ϵ}^{2}}{( sR + {τ}^{2} ) {σ}_{p}^{2} / N} . & [ 124 ]\end{matrix}$$

### Example 7

### Within-Family Expected Allele Frequency Difference

A balanced within-family design is described in which each family contributes s′ sibs to the upper pool and s′ sibs to the lower pool. We derive an analytical expression for the expected allele frequency difference and NCP for a related design in which sib phenotypic values are re-expressed as the sum of a family component (the mean phenotypic value for a family) and an individual component (the difference between the phenotypic value of a sib and the family mean), and a fraction f equal to s′/s of the sibs with the most extreme high and low individual components of phenotypic value are selected for the upper and lower pools. In the text, we show that the analytical expression is accurate when compared to a numerical calculation.

The non-shared phenotypic component for sib i of family k is denoted X′ₖᵢ,

$${X}_{k1}^{′} = {X}_{k1} - {X}_{k \cdot } = {Y}_{k1}^{′} + {μ}_{k1}^{′} , \qquad [ 125 ]$$

where

$$\begin{matrix}{Y}_{k1}^{′} ∼ N ( 0 , {σ}_{R}^{2} - ( 1 / s ) [ {σ}_{R}^{2} + ( s - 1 ) ( t - r {σ}_{A}^{2} - u {σ}_{D}^{2} ) ] ) \\ = N [ 0 , ( 1 - T ) {σ}_{R}^{2} ] ,\end{matrix} \qquad [ 126 ]$$

μ′ₖᵢ=μ(Gₖᵢ)−μ_{k•}, [127]

and the mean values X_{k•} and μ_{k•} have the same meaning as before.

Using f to represent the pooling fraction n/N,

$$\begin{matrix}f = {\sum }_{G} P ( G ) {\int }_{{X}_{b}}^{∞} ⅆ {X [ 2 π ( 1 - T ) {σ}_{R}^{2} ]}^{- 1 / 2} \\ {exp [ - X - {μ}_{1}^{′} )}^{2} / 2 ( 1 - T ) {σ}_{R}^{2} ] ,\end{matrix} \qquad [ 128 ]$$

where G represents the genotypes G₁, G₂, . . . , Gₛ for a sib-ship of size s, P(G) is the corresponding joint probability distribution normalized to 1, λ₁′ is μ(G₁)−μ_{G}, and, by symmetry, only the first sib need be considered. Expanding the exponential and keeping terms through order μ_{G},

$$\begin{matrix}f = {\sum }_{G} P ( G ) {\int }_{{X}_{b}}^{∞} ⅆ {X [ 2 π ( 1 - T ) {σ}_{R}^{2} ]}^{- 1 / 2} \\ ( 1 + {μ}_{1}^{′} X / ( 1 - T ) {σ}_{R}^{2} ) exp [ - {X}^{2} / 2 ( 1 - T ) {σ}_{R}^{2} ] \\ = Φ [ - {X}_{U} / {( 1 - T )}^{1 / 2} {σ}_{R} ]\end{matrix} \qquad [ 129 ]$$

Inverting this equation yields −(1−T)^{1/2}σ_{R}Φ⁻¹(f) as the pooling threshold.

With the threshold determined, the expected allele frequency for the upper pool, E(\hat{p}_{U}), is

$$\begin{matrix}E ( {\hat{p}}_{u} ) = ( 1 / f ) {\sum }_{G} P ( G ) {p}_{1} {\int }_{{X}_{b}}^{∞} ⅆ {X [ 2 π ( 1 - T ) {σ}_{R}^{2} ]}^{- 1 / 2} \\ exp [ - {( X - {μ}_{1}^{′} )}^{2} / 2 ( 1 - T ) {σ}_{R}^{2} ] ,\end{matrix} \qquad [ 130 ]$$

where p₁ is the allele frequency for sib 1. Again keeping terms through order μ_{G},

$$\begin{matrix}E ( {\hat{p}}_{u} ) = ( 1 / f ) {\sum }_{G} P ( G ) {p}_{1} {\int }_{{X}_{i}}^{x} ⅆ {X [ 2 π ( 1 - T ) {σ}_{R}^{2} ]}^{- 1 / 2} \\ [ 1 + {μ}_{1}^{′} X / ( 1 - T ) {σ}_{R}^{2} ] exp [ - {X}^{2} / 2 ( 1 - T ) {σ}_{R}^{2} ] \\ = p + [ y / {( 1 - T )}^{1 / 2} {σ}_{R} f ] E ( {p}_{1} {μ}_{1}^{′} ) .\end{matrix} \qquad [ 131 ]$$

The final expectation required is

$$\begin{matrix}E ( {p}_{1} {μ}_{1}^{′} ) = E [ {p}_{1} \cdot ( {μ}_{s} - {s}^{- 1} {\sum }_{j = 1}^{s} {μ}_{1} ) ] \\ = {σ}_{p} {σ}_{A} \cdot \{ 1 - {s}^{- 1} [ 1 + ( s - 1 ) r ] \} \\ = ( 1 - R ) {σ}_{p} {σ}_{A} ,\end{matrix} \qquad [ 132 ]$$

and the expected allele frequency for the upper pool is

E(\hat{p}_{U})=p+y[(1−R)/f(1−T)^{1/2}](σₚσ_{A}/σ_{R}). [133]

By symmetry, the lower pool has an offset of equal magnitude and opposite direction, yielding an expected allele frequency difference of

$$E ( {\hat{p}}_{U} - {\hat{p}}_{I} ) = 2 \frac{y ( 1 - R ) {σ}_{p} {σ}_{A}}{{f ( 1 - T )}^{1 / 2} {σ}_{R}} . \qquad [ 134 ]$$

Dividing the square of the expected allele frequency difference by its variance gives the NCP for the between-family design,

$$\begin{matrix}NCP = \frac{N {σ}_{A}^{2}}{{σ}_{R}^{2}} \cdot \frac{( s - 1 ) ( 1 - R )}{s ( 1 - T )} \cdot \frac{1}{1 + {τ}^{2} / ( 1 - r )} \cdot \frac{2 {y}^{2}}{f + {f}^{2} {κ}^{2}} , \\ with & [ 135 ] \\ {κ}^{2} = \frac{{ϵ}^{2}}{( 1 - r + {τ}^{2} ) {σ}_{p}^{2} / N} . & [ 136 ]\end{matrix}$$

### Example 8

### Within-Family Expected Allele Frequency Difference for Sib-Pairs

For the within-family pool design, we restrict attention to sib-pairs. For each family k, half the phenotype difference between sibs 1 and 2 is denoted ΔXₖ=(Δₖ₁−Xₕ₂)/2. In terms of the variance components model,

ΔXₖ=ΔYₖ+Δμₖ, 137]

where

$$Δ {Y}_{k} ∼ N [ 0 , ( {σ}_{R}^{2} - t + r {σ}_{t}^{2} + u {σ}_{D}^{2} ) / 2 ] = N [ 0 , ( 1 - T ) {σ}_{R}^{2} ] \qquad [ 138 ]$$

and

Δμₖ=[μ(Gₖ₁)−μ(Gₖ₂)]/2. [139]

The definition of Tin the middle equation is identical to that for the between-family design with s=2. Families are ranked by |ΔXₖ|, and the n families having the largest magnitude are identified as the source of the 2n individuals to be pooled. The threshold magnitude is denoted X_{T} and is related to the pooling fraction f through the equation

$$\begin{matrix}f = ( 1 / 2 ) {\sum }_{G} P ( G ) [ {\int }_{- ∞}^{- {X}_{I}} + {\int }_{{X}_{I}}^{∞} ] ⅆ {X [ 2 π ( 1 - T ) {σ}_{R}^{2} ]}^{- 1 / 2} \\ exp [ - {( X - Δ {μ}_{G} )}^{2} / 2 ( 1 - T ) {σ}_{R}^{2} ]\end{matrix} \qquad [ 140 ]$$

The leading factor of (½) indicates that only 1 sib is selected for each pool, and the term Δμ_{G} corresponds to the term Δμₖ in the variance components model for G=(G₁,G₂).

While it is possible to invert this equation numerically to obtain X_{T} as a function of f, an analytical approximation derived by expanding the exponential to lowest order in Δμ_{G},

$$exp [ - {( X - Δ {μ}_{G} )}^{2} / 2 ( 1 - T ) {σ}_{R}^{2} ] \approx \\ [ 1 + X {Δμ}_{G} / ( 1 - T ) {σ}_{R}^{2} ] exp [ - {X}^{2} / 2 ( 1 - T ) {σ}_{R}^{2} ] \qquad [ 141 ]$$

is very accurate for QTLs with small effect. The result for the pooling fraction is

f=Φ[−X₁/(1−T)^{1/2}σ_{R}]. [142]

The expected allele frequency difference between pools is

$$E ( {\hat{p}}_{ij} - {\hat{p}}_{j} ) = ( 1 / 2 f ) {\sum }_{G} P ( G ) [ p ( {G}_{1} ) - p ( {G}_{2} ) ] \times [ - {\int }_{- ∞}^{- Xi} + {\int }_{Xi}^{∞} ] ⅆ {X [ 2 π ( 1 - T ) {σ}_{R}^{2} ]}^{- 1 / 2} exp [ - {( X - {Δμ}_{G} )}^{2} / 2 ( 1 - T ) {σ}_{R}^{2} ] \qquad [ 143 ]$$

and may be calculated numerically. Alternately, the low-order expansion for the exponential may be inserted to yield

$$E ( {\hat{p}}_{ij} - {\hat{p}}_{i} ) = ( 1 / 2 f ) {\sum }_{G} P ( G ) [ p ( {G}_{1} ) - p ( {G}_{2} ) ] \cdot 2 yΔ {μ}_{G} / {( 1 - T )}^{1 / 2} {σ}_{R} , \qquad [ 144 ]$$

where y is the height of the standard normal probability density when the cumulative probability is f.

The genotype-dependent sum is

$${\sum }_{G} P ( G ) [ p ( {G}_{1} ) - p ( {G}_{2} ) ] Δ {μ}_{G} = ( 1 / 2 ) {\sum }_{G} P ( G ) \{ p ( {G}_{1} ) μ ( {G}_{1} ) + p ( {G}_{2} ) μ ( {G}_{2} ) - p ( {G}_{1} ) μ ( {G}_{2} ) - p ( {G}_{2} ) μ ( {G}_{1} ) \} = ( I - r ) {σ}_{p} {σ}_{1} = 2 ( 1 - R ) {σ}_{p} {σ}_{A} \qquad [ 145 ]$$

where R has the same definition as for the between-family design. Inserting this into the previous equation yields

$$E ( {\hat{p}}_{ij} {\hat{- p}}_{i} ) = 2 y ( 1 - R ) {σ}_{P} \frac{{σ}_{A}}{{f ( 1 - T )}^{1 / 2} {σ}_{R}} \qquad [ 146 ]$$

for the expected allele frequency difference. Recalling the variance of the estimator,

$$Var ( {\hat{p}}_{ij} - {\hat{p}}_{i} ) = 4 ( 1 - R ) {σ}_{p}^{2} / Nf + 2 {τ}^{2} {σ}_{p}^{2} / Nf + 2 {ϵ}^{2} \qquad [ 147 ]$$

yields for the NCP the value

$$\begin{matrix}NCP = \frac{N {σ}_{4}^{2}}{{σ}_{R}^{2}} \cdot \frac{( 1 - R )}{2 ( 1 - T )} \cdot \frac{1}{1 + {τ}^{2} / ( 1 - r )} \cdot \frac{2 {y}^{2}}{f + {f}^{2} {κ}^{2}} , \\ with & [ 148 ] \\ {κ}^{2} = \frac{{ϵ}^{2}}{( 1 - r + {τ}^{2} ) {σ}_{p}^{2} / N} . & [ 149 ]\end{matrix}$$

### Example 9

### Analytical Fit for the Optimal Pooling Fraction

The pooling fraction is optimized to maximize the value of the information retained by the NCP, which is equivalent to maximizing the value of

I=2y²/(f+f²κ²). [150]

Both y and/may be expressed in terms of a normal deviate z,

y=exp(−z²/2)/\sqrt{2π}, [151]

and

f=Φ(−z), [152]

where the use of −z in the definition of f provides z>0 for convenience. Taking the derivative of 1 with respect to z and dividing by non-zero terms,

y·(1+2fκ²)−2zf·(1+fκ²)=0 [153]

yields the optimum; we have used dy/dz=−yz and df/dz=−y.

When κ² is large, z is also large, and f may be replaced by its asymptotic expansion for large z,

f=y·(z⁻¹−z⁻³). [154]

With this substitution, the optimum satisfies.

z³/2yκ²=1. [155]

Taking the natural logarithm of both sides and equating exponents,

J(z)=z²/2+3 ln z−ln(κ²{square root over (2/π))}). [156]

When κ and z are both large, the term proportional to ln z is asymptotically small, and the asymptotic result for z is

z˜B(κ)≡\sqrt{(2κ⁴/π)}. [157]

An improved fit is obtained by perturbation theory by writing

z=B(κ)[1+b(κ)], [158]

where

$${lim}_{A \to ∞} b ( κ ) = 0.$$

Substituting this expression for z into J(z) and simplifying,

B²b+3 ln[B(1+b)]=0, [159]

which gives the asymptotic form b=(3/B²)ln B, or

z˜B−(3/B)ln B. [160]

This form provides a good fit when κ is much larger than 1 but not for smaller values. Since the asymptotic behavior for large κ is not affected by introducing terms of lower order in κ, the fit can be improved for small κ without affecting the fit at large κ by writing

z=A−(3/A)ln A+a₁, [161]

where

A(κ)=\sqrt{a₂+ln(1a₃κ²+278 ⁴π)}. [162]

The constants a₁, a₂, and a₃ are then selected to fit the exact numerical results at particular values of κ. Fitting the results 7=0.612 at κ=0 and z=0.8047 at κ=1 provides the particular parameters

a₁=−0.067, a₂=2, a₃=3. [163]

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of starting genetic material, clone of interest, or library type is believed to be a matter of routine for a person of ordinary skill in the art with knowledge of the embodiments described herein. Also routine are choice of selection module, pooling module, measuring module, association detection module, and reporting module. Other aspects, advantages, and modifications considered to be within the scope of the following claims. The claims presented are representative of the inventions disclosed herein. Other, unclaimed inventions are also contemplated. Applicants reserve the right to pursue Such inventions in later claims.

What is claimed is:

1. A system, said system comprising:
at least one selection module for selecting individuals with at least one pre-determined phenotypic value;
at least one pooling module that pools genetic materials of the selected individuals into at least one pool;
at least one measuring module that measures a frequency of at least one allele of each pool;
at least one association detection module for detecting an association between at least one genetic locus and at least one phenotype by measuring an allele frequency difference between pools; and
at least one reporting module that presents the results of the association detection;
wherein said system detects in a population of individuals at least one association between at least one genetic locus and at least one phenotype, where two or more alleles occur at each genetic locus, and where the system optimizes at least one parameter for detection of the association.

2. The system of claim 1 further comprising a validation module that validates the detected association, the validation module comprising genotyping at least one genetic marker for at least one detected allele from the association detection module with a plurality individuals in the original population.

3. The system of claim 1, wherein a difference in frequency of occurrence of the specified allele is associated with a plurality of errors.

4. The system of claim 3, wherein the error is due to an unequal contribution of a DNA concentration of individuals to the pool.

5. The system of claim 3, wherein the error is due to informalities in measurement.

6. The system of claim 1, wherein the predetermined phenotypic value comprises a value having a lower limit and an upper limit, wherein the lower limit has a value set so that the pool of a first selection has a value between about the highest 37% of the population to about the highest 19% of the population, and wherein the predetermined upper limit has a value set so that the pool of a second selection has a value between about the lowest 37% of the population to about the lowest 19% of the population.

7. The system of claim 6, wherein the value of the predetermined lower limit is set so that the pool of the first selection has a value of about the highest 27% of the population and the predetermined upper limit is set so that the pool of the second selection has a value of about the lowest 27% of the population.

8. The system of claim 1, wherein the population includes individuals who are classified into classes.

9. The system of claim 8, wherein the classes are based on an age group, a gender, a race or an ethnic origin.

10. The system of claim 8, wherein all the members of a class are included in the pool.

11. The system of claim 1, wherein the association detection module detects a genetic basis of disease predisposition.

12. The system of claim 11, wherein the genetic locus that is analyzed for determining the genetic basis of disease predisposition contains a single nucleotide polymorphism.

13. The system of claim 1, wherein the system optimizes the association detection by determining the minimum number of individuals from the population that is required for detecting the association using a non-centrality parameter.

14. The system of claim 13, wherein the non-centrality parameter is defined as,
$$NCP = \frac{NR {σ}_{4}^{2}}{sT {σ}_{R}^{2}} \cdot \frac{1}{1 + {τ}^{2} / sR} \cdot \frac{2 {y}_{+}^{2}}{{f}_{+} + {f}_{+}^{2} {κ}_{+}^{2}} , wherein$$
$$R = ( 1 / s ) [ 1 + ( s - 1 ) r ] , \\ T = ( 1 / s ) [ {σ}_{R}^{2} + ( s - 1 ) ( t - r {σ}_{4}^{2} - u {σ}_{1 )}^{2} ) ] \approx ( 1 / s ) [ 1 + ( s - 1 ) t ] , and$$
$${κ}_{+}^{2} = {ϵ}^{2} / [ ( sR + {τ}^{2} ) ( {σ}_{p}^{2} / N ) ] .$$

15. The system of claim 1, wherein the association detection module is used in a within-family design to detect the association between at least one genetic locus and at least one phenotype.

16. The system of claim 1, wherein the association detection module is used in a between-family design to detect the association between at least one genetic locus and at least one phenotype.

17. A method of detection, the method comprising:
selecting individuals with at least one predetermined phenotypic value;
pooling genetic materials of selected individuals into at least one pool;
measuring a frequency of at least one allele of each pool;
detecting an association between at least one genetic locus and at least one phenotype by measuring an allele frequency difference between pools; and
presenting a result of the association detection;
wherein said method detects an association in a population of individuals between one or more genetic locus and one or more phenotypes, where two or more alleles occur at each genetic locus, and wherein the system optimizes one or more parameter s for detection of the association.

18. The method of claim 17 further comprising validating the association by genotyping genetic markers for at least one detected allele from the association detection module with a plurality of individuals in the original population.

19. The method of claim 17, wherein the difference in frequency of occurrence of the specified allele is associated with a plurality of errors.

20. The method of claim 19, wherein the error is due to an unequal contribution of a DNA concentration from at least one individual to the pool.

21. The method of claim 19, wherein the error is due to informalities in measurement.

22. The method of claim 17, wherein the predetermined phenotypic value comprises values having a lower limit and an upper limit, wherein the lower limit has a value set so that the pool of a first selection has a value between about the highest 37% of the population to about the highest 19% of the population, and wherein the predetermined upper limit has a value set so that the pool of a second selection has a value between about the lowest 37% of the population to about the lowest 19% of the population.

23. The method of claim 22, wherein the value of the predetermined lower limit is set so that the pool of the first selection has a value of about the highest 27% of the population and the predetermined upper limit is set so that the pool of the second selection has a value of about the lowest 27% of the population.

24. The method of claim 17, wherein the population includes individuals who are classified into at least one class.

25. The method of claim 24, wherein the classes are based on an age group, a gender, a race or an ethnic origin.

26. The method of claim 24, wherein all members of the class are included in the pool.

27. The method of claim 17, wherein the association detection module detects the genetic basis of a disease predisposition.

28. The method of claim 27, wherein the genetic locus that is analyzed for determining the genetic basis of the disease predisposition contains a single nucleotide polymorphism.

29. The method of claim 17, wherein the method optimizes the association detection by determining the minimum number of individuals from the population required for detecting the association when using a non-centrality parameter.

30. The method of claim 29, wherein the non-centrality parameter is defined as,
$$NCP = \frac{NR {σ}_{A}^{2}}{sT {σ}_{R}^{2}} \cdot \frac{1}{1 + {τ}^{2} / sR} \cdot \frac{2 {y}_{+}^{2}}{{f}_{+} + {f}_{+}^{2} {κ}_{+}^{2}} , wherein$$
$$R = ( 1 / s ) [ 1 + ( s - 1 ) r ] , \\ T = ( 1. s ) [ {σ}_{R}^{2} + ( s - 1 ) ( t - r {σ}_{4}^{2} - u {σ}_{D}^{2} ) ] \approx ( 1 / s ) [ 1 + ( s - 1 ) t ] , and$$
$${κ}_{+}^{2} = {ϵ}^{2} / [ ( sR + {τ}^{2} ) ( {σ}_{p}^{2} / N ) ] .$$

31. The method of claim 17, wherein the association detection module is used in a within-family design to detect the association between at least one genetic locus and at least one phenotype.

32. The method of claim 17, wherein the association detection module is used in a between-family design to detect the association between at least one genetic locus and at least one phenotype

33. A system of detection, said system comprising:
a selection means for selecting individuals with at least one pre-determined phenotypic value;
a pooling means that pools genetic material from the selected individuals into at least one pool;
a measuring means that measures the frequency of at least one allele from each pool of selected individuals;
an association detection means for detecting an association between at least one genetic locus and at least one phenotype by measuring the allele frequency difference between pools; and
a reporting means that present the results of the association detection;
wherein said system detects the association in a population of individuals between at least one genetic locus and at least one phenotype, where two or more alleles occur at each genetic locus, and where the system optimizes at least one parameter for detection of the association, the system.

34. A processor readable medium, said processor readable medium comprising:
a first processor readable program code for causing a processor to select individuals with a pre-determined phenotypic value;
a second processor readable program code for causing a processor to pool genotype-related data from the selected individuals into at least one pool;
a third processor readable program code for causing a processor to measure a frequency of one or more alleles in each pool;
a fourth processor readable program code for causing a processor to detect an association between at least one genetic locus and at least one phenotype by measuring an allele frequency difference between pools; and
a fifth processor readable program code for causing a processor to present the results of the association detection;
wherein said processor readable code embodied therein detects an association in a population of individuals between at least one genetic locus and at least one phenotype, where two or more alleles occur at each genetic locus, and where the system optimizes at least one parameter for detection of the association, the processor usable medium.

35. The processor readable medium of claim 34, wherein the second processor readable program code causes the processor to pool genotype-related data from two or more preexisting pools of genotype-related data for sub-populations of selected individuals into at least one larger pool.
